(19) United States
(12) Patent Application Publication
(10) Pub. No.: US 2010/0310543 A1
(43) Pub. Date: Dec. 9, 2010

(54) METHOD OF PREVENTING AND TREATING ACUTE BRAIN PATHOLOGIES

(75) Inventors: Melissa Farinelli, Basel (CH); Isabelle Mansuy, Zurich (CH); Frabice Heitz, Eschau (DE)

Correspondence Address: MH2 TECHNOLOGY LAW GROUP, LLP, 1951 KIDWELL DRIVE, SUITE 550, TYSONS CORNER, VA, 22182, US

(73) Assignee: UNIVERSITY OF ZURICH, Zurich (CH)

(21) Appl. No.: 12/667,733

(22) PCT Filed: Jul. 7, 2008
(86) PCT No.: PCT/EP2008/005522

§ 371 Date: Aug. 11, 2010

(30) Foreign Application Priority Data
Jul. 6, 2007 (EP) 07013277.4

(51) Int. Cl. A61K 38/45; A61K 31/22; G01N 33/53; A61K 38/16; A61P 25/00; A61P 9/10; A61P 25/08

(52) U.S. Cl. 424/94.5; 514/551; 435/7.1; 514/17.7

(57) ABSTRACT

Described are novel means in the treatment of cerebral neurological conditions such as ischemic insult of the brain. Furthermore, a kit for a non-radioactive protein serine/threonine phosphatase activity assay is provided, which is capable of detecting and distinguishing the activity of PP1, PP2A, and calcineurin (PP2B).

### FIELD OF THE INVENTION

The present invention relates to the technical field of neurological disorders and methods for the treatment of the same. More specifically, the present invention pertains to the treatment of disorders mediated by perturbed protein phosphorylation in particular, involving protein phosphatase 1 (PP1). A further object of the present invention is to provide a kit for a non-radioactive protein serine/threonine phosphatase activity assay, which is capable of detecting and distinguishing the activity of PP1, PP2A, and calcineurin (PP2B).

### BACKGROUND OF THE INVENTION

Cerebral ischemia often leads to excitotoxic damage resulting from transient deprivation of oxygen and nutrients in the brain. Such deprivation causes a dramatic increase in neuronal excitation due to enhanced glutamate release and a toxic rise in intracellular calcium (Ca²⁺) in several cellular compartments including mitochondria (Arundine and Tymianski, 2004; Olney and Sharpe, 1969). Affected regions of the brain are often irreversibly injured and have long-lasting functional impairments. Because ischemia is highly detrimental to brain functions, much work has been done to better understand the underlying molecular mechanisms and attempt to develop neuroprotective therapies. One of the primary strategies has focused on glutamate receptor (NMDA or AMPA) antagonists (Aarts and Tymianski, 2003). Many of these strategies however, were not successful in clinical trials because of major side effects of the selected drugs (Hoyte et al., 2004). An alternative approach currently receiving consideration is to target intracellular pathways downstream from glutamate receptors. These pathways are however still not well understood and need to be studied in greater detail before any new therapeutic approach can be envisaged (Aarts and Tymianski, 2003; Legos et al., 2002).

One of the major features of ischemic insult is the occurrence of massive intracellular Ca²⁺ overload due to hyperactivation of the NMDA receptor. This overload dramatically alters Ca²⁺-dependent signaling and Ca²⁺ buffering systems (Arundine and Tymianski, 2003; Lipton, 1999; Sattler and Tymianski, 2000; Sugawara et al., 2004). It also perturbs the redox activity of the mitochondrial respiratory chain and induces the production of free radicals by activating synthesizing enzymes such as neuronal nitric oxide synthase and cyclooxygenase, leading to apoptosis (Lipton, 1999; Sugawara et al., 2004). Impaired Ca²⁺ homeostasis further drastically alters the activity of Ca²⁺-sensitive protein kinases and phosphatases. Such alterations have a severe impact on the outcome of excitotoxicity because protein kinases and phosphatases critically regulate multiple substrates involved in cell survival and cell death pathways in nerve cells. For instance, a change in the balance of activity between the Ca²⁺-dependent protein kinase CaMKII, the protein kinase C (PKC) (Aronowski et al., 2000; Onodera et al., 1995) and the protein phosphatase calcineurin (CN or PP2B) in favor of CN, was shown to induce excessive dephosphorylation of pro-apoptotic proteins such as Bad, a Bcl-2 family member (Asai et al., 1999; Wang et al., 1999), and delayed neuronal cell death. In contrast, inhibition of CN by the immunosuppressants FK506 or CsA prevents neuronal degeneration, reduces infarct size and rescues motor functions after experimental stroke in the rat (Sharkey and Butcher, 1994; Sharkey et al., 1996) (Morioka et al., 1999). These findings suggested a potential role for CN in the mechanisms of cell death however, the effects of immunosuppressants formerly attributed to CN inhibition are now recognized to be mediated by the immunophilins FKBP12 and cyclophilin A and not CN itself (Kaminska et al., 2004; Klettner et al., 2001). Further, CN inhibition was also recently shown to abolish neuroprotection induced by nicotine after glutamate-mediated excitotoxicity in primary cortical cultures, suggesting a positive effect of CN (Stevens et al., 2003). The results indicate that CN has both neuroprotective and apoptotic functions that may depend on the nature, kinetic or extent of the injury.

### SUMMARY OF THE INVENTION

The present invention relates to the use of agents capable of modulating protein phosphorylation in the treatment, amelioration and prevention, respectively, of cerebral neurological disorders, in particular disorders associated with ischemic insult or related diseases with an insult of the brain. In particular, the present invention makes use of the surprising finding that protein phosphatase 1 (PP1)-dependent pathways and plasticity control the mechanisms of recovery from ischemic insult in vitro and in vivo

Besides the impact of the findings obtained in accordance with the present invention on approaching insults of the brain and disorders related thereto, the present invention also provides novel diagnostic markers for the diagnosis of such disorders, i.e. PP1. In this context, the present invention also pertains to diagnostic compositions and kits for use in corresponding diagnostic methods employing genes and gene products involved in phosphorylation events as a diagnostic marker for an acute neurological condition.

Other embodiments of the invention will be apparent from the description that follows.

### BRIEF DESCRIPTION OF THE DRAWINGS

FIG. 1 Pharmacological inhibition of PP1 by OA or tautomycin reduces f-EPSP recovery after transient OGD. a) Representative graph showing the effect of transient OGD on f-EPSP slope in area CA1 in acute hippocampal slices from control mice. Insets show representative traces (1) before, (2) during and (3, 4) after OGD. The rebound observed after 25-30 min may result from the re-activation of ATPase (Lust et al., 2002). b) The time course and dose-dependent effect of OA at 0.1, 1, 10 or 100 nM, and 1 μM (squares) on f-EPSP slope following transient OGD. Vehicle, DMSO (black dots). c) Mean f-EPSP slope (over the last 10 min of recording) for each OA dose showing a significant reduction in recovery in slices treated with 100 nM and 1 μM OA. d) Time course and dose-dependent effect of tautomycin at 1, 5, 10 and 100 nM (squares) on f-EPSP slope following transient OGD. Vehicle, DMSO (black dots). e) Mean f-EPSP slope (over the last 10 min of recording) for each tautomycin dose used in d). Statistical significance compared to baseline †p<0.05, †††p<0.001; compared to No drug control slices *p<0.05, **p<0.005, ***p<0.001.

FIG. 2: Genetic inhibition of PP1 impairs f-EPSP slope recovery after OGD. a) Reduced f-EPSP slope recovery in mutant slices expressing I-1* compared to control slices. Insets show representative traces before (1, 3) and after (2, 4) OGD in control (1, 2) and mutant (3, 4) slices. b) Mean f-EPSP slope (over the last 10 min of recording) showing a significant reduction in f-EPSP slope recovery in the mutant slices compared to control slices. c) Input-output curve and d) PPF before OGD are similar in control and mutant slices indicating normal basal synaptic transmission. Statistical significance compared to baseline: †††p<0.001; compared to control: **p<0.005.

FIG. 3: The induction of LTP 10 min but not 30 min prior to OGD reduces f-EPSP slope recovery in hippocampal slices. a) Representative LTP induced by a single 1-sec train at 100 Hz: f-EPSP slope 10 min after HFS, 138.8±3.3% of baseline; 30 min after HFS, 130.8±2.9% of baseline, n=18. This form of LTP is blocked by 1 μM OA (Blitzer et al., 1998; Morishita et al., 2001; Thiels et al., 1998; Winder and Sweatt, 2001). Insets show representative traces before (1) and after (2) LTP induction. b) LTP was induced 10 min (LTP+10 min, gray squares) or 30 min (LTP+30 min, open squares) prior to OGD and f-EPSP slopes were compared to OGD alone (No LTP, black dots). c) Mean f-EPSP slope (over the last 10-min of recording) in OGD with no LTP, or combined with either LTP+10 min or LTP+30 min. LTP+10 min versus no LTP, ***p<0.005; No LTP, LTP+10 min or LTP+30 min versus baseline, †††p<0.0005 and †p<0.05. d) Level of PP1 inhibition (% baseline) either immediately (OGD 0 min) or 1 hour (OGD 1 hr) after OGD in CA1 of hippocampal slices not subjected to LTP (No LTP) or preceded by LTP+10 min. Statistical significance compared to baseline: †p<0.05, ††p<0.01, †††p<0.001; compared to No LTP: *p<0.05.

FIG. 4: LTD induction 10 min but not 30 min prior to OGD improves f-EPSP slope recovery in control hippocampal slices. a) Representative LTD induced by 10 min stimulation at 2 Hz (paired pulase): f-EPSP slope 10 min after LFS, 55.7±3.0% of baseline; 30 min after LFS, 82.6±1.7% of baseline, n=6. Insets show representative traces before (1) and after (2) LTD induction. b) LTD was induced 10 min (LTD+10 min, gray squares) or 30 min (LTD+30 min, open squares) prior to OGD and f-EPSP slope was compared with OGD alone (No LTD, black dots). c) Mean f-EPSP slope (over the last 10 min of recording) in OGD with No LTD, LTD+10 min or LTD+30 min. d) Level of PP1 inhibition (% baseline) either immediately (OGD 0 min) or 1 hour (OGD 1 hr) after OGD in CA1 of hippocampal slices not subjected to LTD (No LTD) or preceded by LTD+10 min. Statistical significance compared to baseline: †p<0.05, ††p<0.01, †††p<0.001; compared to No LTD: *p<0.05, **p<0.01.

FIG. 5: Full inhibition of PP1 blocks the beneficial effect of LTD on f-EPSP recovery. a) Normal LTD in control slices treated with 1 nM tautomycin. b) Impaired LTD in I-1* mutant slices treated with 1 nM tautomycin but normal LTD in the absence of the drug. The reduced LTD reported previously in I-1* mutant mice was observed after a 10 min stimulation at 2 Hz (Jouvenceau et al., 2006), which is different from the paired pulse stimulation used here (see Supplementary Methods). c) f-EPSP slope recovery is prevented by 1 nM tautomycin after LTD+10 min and OGD (open circles) but not in the absence of the drug (black circles) in I-1* mutant slices. Open squares, OGD in controls; open triangle, LTD in controls. d) Mean f-EPSP slope (over the last 10 min of recording) for LTD without (No drug) or with 1 nM tautomycin (Tauto) in control and mutant slices. e) Mean f-EPSP slope (over the last 10 min of recording) for OGD in control slices (No LTD) or mutant slices subjected to LTD+10 min and OGD without (LTD) or with (LTD+Tauto) 1 nM tautomycin. Statistical significance compared to respective No drug conditions: *p<0.05, **p<0.005.

FIG. 6: Genetic inhibition of PP1 aggravates brain infarct and increases disseminate neuronal injury following MCAO. a, b). Representative cresyl violet staining of coronal brain sections (a), and bar graph indicating mean infarct volume (b) 24 hours following 90 min MCAO and reperfusion in I-1* mutant mice and control littermates. Scale bar=2 mm. d, e). Representative TUNEL staining (d) and bar graph indicating mean density of DNA-fragmented cells (e) in striatum 72 hours following 30 min MCAO and reperfusion in I-1* mutant mice and control littermates. Scale bar=100 μm. e, f) Laser doppler flowmetry during 90 min (e) or 30 min (f) MCAO followed by 30 min reperfusion indicating similar level of perfusion in I-1* mutant mice and control littermates. *p<0.05.

FIG. 7: PP1 inhibition increases ERK1/2 phosphorylation after MCAO in vivo and OGD in vitro. Western blots (top panels) and quantified bar graphs (middle and bottom panels) showing a, b) total and phosphorylated ERK1 and ERK2 and c, d) total and phosphorylated JNK1 and JNK2. a, c) Contralateral (none) and ipsilateral (MCAO) homogenates from striatum in I-1* mutant mice (black) and control littermates (white) after 30 min MCAO and 72 hr reperfusion (n=6/group). b, d) Slices from control mice subjected to OGD (OGD) or no OGD (none) treated with 3 nM tautomycin (black) or vehicle (white). Statistical significance comparing no MCAO/OGD (none) to other groups, †p<0.05; MCAO in control mice or OGD in slices not treated with tautomycin (No drug) to no MCAO/OGD, MCAO in mutant mice or OGD in slices treated with tautomycin (Tautomycin), #p<0.05; no MCAO to MCAO in mutant mice, and no OGD to OGD in slices treated with tautomycin, *p<0.05.

FIG. 8: PP1 inhibition decreases Bcl-X_{L} expression and increases caspase-3 activation after MCAO. Western blots (top panels) and quantified bar graphs (middle and bottom panels) of a) Bcl-X_{L} and b) caspase-3 in contralateral (none) and ipsilateral (MCAO) homogenates from striatum in I-1* mutant mice and control littermates after 30-min MCAO and 72-hour reperfusion (n=6/group). Statistical significance comparing no MCAO (none) to other groups, †p<0.05; MCAO in control mice to no MCAO or MCAO in mutant mice, #p<0.05; no MCAO to MCAO in mutant mice, *p<0.05.

FIG. 9: Two-pathway recording of the detrimental effect of LTP and the beneficial effect of LTD on f-EPSP recovery after transient OGD in hippocampal slices. a) LTP was induced in one pathway by a 1 sec train at 100 Hz applied to Schaffer collaterals 10 min prior to OGD (LTP+10 min pathway) while the other pathway was not stimulated (No LTP pathway). b) Mean f-EPSP slope (over the last 10 min of recording) after transient OGD in the control pathway (No LTP), and the pathway subjected to LTP (LTP+10 min). c) LTD was induced in one pathway by 10 min of paired pulse stimulation at 2 Hz applied to Schaffer collaterals 10 min prior to OGD (LTD+10 min pathway) while the other pathway was not stimulated (No LTD pathway). d) Mean f-EPSP slope (over the last 10 min of recording) after transient OGD in the control pathway (No LTD), and the pathway subjected to LTD (LTD+10 min). Statistical significance compared to baseline, †††p<0.001; non-stimulated pathway to LTP or LTD pathway, ***p<0.001.

FIG. 10: PP2A inhibition following OGD is not changed by LTP or LTD. Level of PP2A inhibition (% baseline) either immediately (OGD 0 min) or 1 hour (OGD 1 hr) after OGD in CA1 of hippocampal slices either a) not subjected to LTP (No LTP) or preceded by LTP+10 min, or b) not subjected to LTD (No LTD) or preceded by LTD+10 min. Statistical significance compared to baseline: †p<0.05.

### DEFINITIONS

Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2.

“Level”, as the term is used herein, generally refers to a gage of, or a measure of the amount of, or a concentration of a transcription product, for instance an mRNA, or a translation product, a protein.

“Activity”, as the term is used herein, generally refers to a measure for the ability of a transcription product or a translation product to produce a biological effect or a measure for a level of biologically active molecules. The terms “level” and/or “activity” as used herein further refer to gene expression levels, gene activity, or enzyme activity.

“Modulator”, as the term is used herein, generally refers to a molecule capable of changing or altering the level and/or the activity of a gene, or a transcription product of a gene, or a translation product of a gene. Preferably, a “modulator” is capable of changing or altering the biological activity of a transcription product or a translation product of a gene. Said modulation, for instance, may be an increase or a decrease in enzyme activity, a change in binding characteristics, or any other change or alteration in the biological, functional, or immunological properties of said translation product of a gene.

“Probes”, as the term is used herein, generally refers to short nucleic acid sequences of the nucleic acid sequences of phosphatases and kinases referred to, described and/or disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample.

“Agent”, “reagent”, or “compound”, as the terms are used herein, generally refer to any substance, chemical, composition, or extract that have a positive or negative biological effect on a cell, tissue, body fluid, or within the context of any biological system, or any assay system examined. They can be agonists, antagonists, partial agonists or inverse agonists of a target. Such agents, reagents, or compounds may be nucleic acids, natural or synthetic peptides or protein complexes, or fusion proteins. They may also be antibodies, organic or inorganic molecules or compositions, small molecules, drugs and any combinations of any of said agents above. Furthermore, as can be inferred from the examples the term “agent” includes physical entities such as irradiation including acoustic, ultraviolet, infrared and laser radiation, thermal energy, electric energy, and the like. They may be used for testing, for diagnostic or for therapeutic purposes.

If not stated otherwise, the terms “compound”, “substance” and “(chemical) composition” are used interchangeably herein and include but are not limited to therapeutic agents (or potential therapeutic agents), food additives and nutraceuticals. They can also be animal therapeutics or potential animal therapeutics.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

Furthermore, the term “subject” as employed herein relates to animals in need of therapy, e.g. amelioration, treatment and/or prevention of an acute brain condition such as stroke or ischemia. Most preferably, said subject is a human.

### General Techniques

For further elaboration of general techniques useful in the practice of this invention, the practitioner can refer to standard textbooks and reviews in cell biology and tissue culture; see also the references cited in the examples. General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Non-viral Vectors for Gene Therapy (Wagner et al. eds., Academic Press 1999); Viral Vectors (Kaplitt & Loewy eds., Academic Press 1995); Immunology Methods Manual (Lefkovits ed., Academic Press 1997); and Cell and Tissue Culture: Laboratory Procedures in Biotechnology (Doyle & Griffiths, John Wiley & Sons 1998). Reagents, cloning vectors and kits for genetic manipulation referred to in this disclosure are available from commercial vendors such as BioRad, Stratagene, Invitrogen, Sigma-Aldrich, and ClonTech.

### DETAILED DESCRIPTION OF THE INVENTION

The present invention generally relates to means and methods for the treatment of neurological disorders and brain diseases in particular. More specifically, as disclosed in Example 4, it could surprisingly be shown that modulation of phosphorylation and dephosphorylation activity, respectively, provides a therapeutic approach for the treatment, amelioration and prevention of impairment of brain functions. The present invention is based on the observation that PP1 dependent pathways and plasticity control the mechanisms of recovery from ischemic insult in vitro and in vivo.

Protein kinases and phosphatases can alter the impact of excitotoxicity resulting from ischemia by concurrently modulating cell death/survival pathways. Without intending to be bound by theory it is, thanks to the experiments performed in accordance with the present invention, believed that the protein phosphatase 1 (PP1), a negative regulator of neuronal signaling and synaptic strength (Mansuy and Shenolikar, 2006; Morishita et al., 2001), critically regulates neuroprotective pathways in the adult brain. When PP1 is inhibited pharmacologically or genetically, recovery from oxygen/glucose deprivation (OGD) in vitro or ischemia in vivo diminishes. In vitro, inducing LTP shortly before OGD similarly impairs recovery, an effect that correlates with strong PP1 inhibition. In accordance with the present invention it could surprisingly be shown that inducing LTD prior to OGD elicits full recovery by preserving PP1 activity, an effect that is abolished by PP1 inhibition. PP1 appears to be coupled with several components of apoptotic pathways, in particular ERK1/2 which activation is increased by PP1 inhibition both in vitro and in vivo. Together, these results reveal a novel mechanism of recovery in the adult brain that depends on PP1, and novel physiological functions for LTP and LTD in the control of brain damage and repair. In addition, Examples 8 and 9 confirm that enhancing PP1 activity by using lentiviral-mediated PP1 overexpression confers a full recovery and tissue protection in hippocampal slices that are transiently deprived of oxygen and glucose (OGD).

Accordingly, the present invention relates to an agent capable of modulating protein phosphatase 1 (PP1) for the treatment, amelioration or prevention of a cerebral neurological condition, especially wherein said neurological condition is due to an excitotoxic injury. The agent in accordance with the present invention can be used for the treatment of neurological disorders, particularly acute neurological conditions, including but not limited to stroke, head or spinal cord trauma, seizure or neurodegenerative disorders or other insults of the central nervous system (CNS) in general. As demonstrated in the examples, activating maintaining, restoring or promoting phosphatase activity, in particular of protein phosphatase 1 (PP1), resulted in the reversion and prevention, respectively, of impairment of synaptic functions of mice which were induced to develop a cerebral neurological disorder.

Hence, the present invention particularly provides a method for treating and preventing acute ischemic insult and disorders associated therewith comprising administering to a subject in need thereof or supposed to become in immediate need a therapeutically effective amount of the agent capable of modulating protein phosphorylation, in particular PP1 activity.

Protein phosphatase 1 (PP1) is a major eukaryotic protein serine/threonine phosphatase that regulates an enormous variety of cellular functions through the interaction of its catalytic subunit (PP1c) with over fifty different established or putative regulatory subunits. Unlike calcineurin (CN), protein phosphatase 1 (PP1), the second most abundant protein serine/threonine phosphatase in the brain, has not been well studied in the context of brain injury but was suggested to have neuroprotective functions. A few reports showed that PP1 inhibition reduces cell survival and induces apoptosis after glutamate-mediated oxidative stress or excitotoxicity in cell culture (Fernandez et al., 1993; Klumpp and Krieglstein, 2002; Nuydens et al., 1998; Runden et al., 1998; Yi et al., 2005), but this potential function has not been investigated in detail. In accordance with the present invention it was examined whether PP1 is involved in the mechanisms of recovery in the adult mouse brain using in vitro and in vivo models of ischemia. Surprisingly, it could be demonstrated that the pharmacological or genetic inhibition of PP1 promotes ischemic damage and prevents recovery following oxygen/glucose deprivation (OGD) in hippocampal slices in vitro, or focal middle cerebral artery occlusion (MCAO) in adult mice; see appended examples. Further, it could be shown that two forms of synaptic plasticity, long-term potentiation (LTP) and long-term depression (LTD) in the adult hippocampus impair and promote recovery, respectively, when induced shortly prior to OGD, and that these effects are associated with opposite regulation of PP1 activity. Indeed, as shown in the examples excitotoxicity during ischemia can be rescued by overexpression of PP1. The results in accordance with the present invention also provide evidence that PP1 is involved in the control of downstream components of apoptotic and cell survival pathways in particular, ERK1/2, both in vitro and in vivo. Overall, these results demonstrate that PP1 and PP1-dependent bidirectional plasticity control the mechanisms of recovery in the adult brain.

The nucleotide and amino acid sequences of the human serine/threonine-protein phosphatases are known in the art and can be obtained via public databases, for example the interne pages hosted by the National Center for Biotechnology Information (NCBI), including the NIH genetic sequence database GeneBank, which also cites the corresponding references available by PubMed Central. For example, the human nucleotide and amino acid sequences of PP1-α catalytic subunits are available under primary Accession number P62136; of the PP1-β catalytic subunit under primary Accession number P62140; and of the PP1-γ catalytic subunit under primary Accession number P36873. The corresponding nucleotide and amino acid sequences of mouse PP1 catalytic subunits are available under Accession numbers P62137, P62141 and P63087. Furthermore, PP1-γ1 and PP1-γ2 catalytic subunits are known which however represent alternatively spliced isoforms generated from a single gene. Most of these target PP1c to specific subcellular locations and interact with a small hydrophobic groove on the surface of PP1c through a short conserved binding motif which is often preceded by further basic residues. Weaker interactions may subsequently enhance binding and modulate PP1 activity/specificity in a variety of ways. Regulation of PP1c in response to extracellular and intracellular signals occurs mostly through changes in the levels, conformation or phosphorylation status of targeting subunits. The mode of action of PP1c complexes facilitates the development of drugs that target particular PP1c complexes and thereby modulate the phosphorylation state of a very limited subset of proteins, for review see, e.g., Cohen, J. Cell Sci. 115 (2002), 241-256. Thus, an agent capable of modulating protein phosphorylation, especially phosphatase modulators can be based on and/or directed to the interaction of the enzyme, e.g., protein phosphatase 1 with any one of its regulatory subunits, most preferably those that bind and are preferably specific for the mentioned binding motif. Such modulators may interfere with complex formation of protein phosphatase complexes and/or targeting the protein phosphatase and complex, respectively, to its native subcellular location. Such modulators are advantageous, since they are more specific than an agent which affects the catalytic activity of the enzyme only. In this context, it is also to be understood that agents useful according to the present invention rather than being directed to the protein phosphatase can be specific for a binding partner of the enzyme such as one of the regulatory subunits which are for example necessary for the enzyme to exert its enzymatic activity and/or correct subcellular location. Activation of PP1 by a small molecule designed to bind to the enzyme's regulatory site is described by Tappan and Chamberlin, Chem. Biol. 15 (2008), 167-174.

In one embodiment of the present invention, the agent for maintaining PP1 activity is capable of inducing long-term depression (LTD), for example electric low-frequency stimulation (LFS) as used in the examples. Techniques of LFS for inducing LTD in human are described in the art, see, e.g. Ellrich and Schorr in Exp. Brain Res. 147 (2002), 549-553 and Brain Res. 996 (2004), 255-258. Furthermore, LFS parameters for humans, for example 1 Hz, 1200 pulses and an intensity (relating to pain threshold I(P)) of 4×I(P) are described and can be evaluated as described in Jung et al., Eur. J. Pain. 2008 May 20; p S1532-2149 [epub ahead of print]. In addition, as described in Udagawa et al., Brain Res. 1124 (2006), 28-36, long-term depression induced by low-frequency stimulation can be enhanced by blocking L-type calcium channels. Alternatively, low-frequency repetitive transcranial magnetic stimulation (rTMS) may be used for inducing LTD as described in Iyer et al., J. Neurosci. 23 (2003), 10867-10872.

A system and an electrical device for applying low-frequency energy, in a range below approximately 10 Hz, to the patient's brain tissue is described in U.S. Pat. No. 6,591,138, the disclosure content of which is incorporated herein by reference. The system comprises an implantable embodiment applying direct electrical stimulation to electrodes implanted in or on the patient's brain and alternatively a non-invasive embodiment causing a magnetic field to induce electrical currents in the patient's brain. Accordingly, the use of the agent in accordance with the present invention present invention also encompasses such a device.

In another embodiment of the present invention, the agent capable of modulating PP1 activity in accordance with the present invention is a drug which can be formulated into a pharmaceutical composition. In this context, PP1 activators are well known in the art and include, for example, ceramide, inhibitor-2 insulin, cdc2-cyclin B, glycogen synthase kinase 3 (GSK3), and p-nitrophenyl phosphate (Aggen et al., 2000; Ceulemans and Bollen, 2007). Preferably, said agent in the composition is present in an amount sufficient to mediate recovery of the brain from an excitotoxic injury.

The pharmaceutical composition of the present invention can be formulated according to methods well known in the art; see for example Remington: The Science and Practice of Pharmacy (2000) by the University of Sciences in Philadelphia, ISBN 0-683-306472. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as dopamine or psychopharmacologic drugs, depending on the intended use of the pharmaceutical composition.

In addition, co-administration or sequential administration of other agents may be desirable. A therapeutically effective dose or amount refers to that amount of the active ingredient sufficient to ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Preferably, the therapeutic agent in the composition is present in an amount sufficient to be capable of maintaining, restoring or promoting PP1 activity.

Meanwhile gene technology-based therapies in the brain have been established for disorders including Alzheimer's disease, Parkinson's disease and brain neoplasms; see for review Wirth and Yla-Herttuala, Adv. Tech. Stand. Neurosurg. 31 (2006), 3-32. For example, lentivirus-mediated gene transfer to the central nervous system in order to provide effective long-term treatment of neurological disorders such as Parkinson's disease, Alzheimer's disease, Huntington's disease, motor neuron diseases, lysosoma storage diseases and spinal injury have been reported; see, for example, the summary in Wong et al., Hum. Gene Ther. 17 (2006), 1-9. Hence, since Examples 8 and 9 demonstrate the beneficial effect of PP1 overexpression in cells and tissue subjected to ischemic conditions gene-therapeutic approaches may be applied in accordance with the of the present invention, including stem cell strategies. For example, human neural stem cells (HNSCs) can be transplanted into the brain, which differentiate into neural cells and significantly improve cognitive functions; see, for example, Sugaya et al., Panminerva Med. 48 (2006), 87-96. Such stem cells may be applied in combination with a gene-therapeutic approach for expressing, e.g., PP1 or a catalytic active subunit thereof. Thus, the findings of the present invention may find their way in various therapeutic approaches, which so far are based on the use of different therapeutic genes.

The person skilled in the art will therefore acknowledge that there are various ways in order to put the present invention into practice and that the means therefore are substantially unlimited. Thus, the agent capable of modulating phosphorylation, in particular PP1 activity, can be of any kind.

In a further aspect, the present invention relates to a method of diagnosis of an acute neurological condition, which comprises:
(a) assaying a sample from a subject for PP1 gene product or activity; and
(b) determining the level of PP1 gene product or activity, wherein an altered level compared to a control indicates the presence of the condition.

In particular, a decreased level of PP1 expression and activity, respectively, compared to a healthy control may be indicative for the presence of the condition. Alternatively, or in addition, a sample from a subject known to suffer from an acute neurological condition such as ischemia in the brain and having an altered level of PP1 gene product or activity, is used as a positive control. In this embodiment, the test subject may be tested positively, if the level of expression or activity of PP1 gene product substantially matches that of the positive control.

In one embodiment, the PP1 gene product is determined by a nucleic acid probe, wherein the nucleic acid is preferably labeled or otherwise modified. Furthermore, microarray and chip technology may be used for determining the level of PP1 gene expression. The use of microarrays in analyzing gene expression is reviewed generally by Fritz et al., Science 288 (2000), 316; Microarray Biochip Technology, www.Gene-Chips.com. An exemplary method is conducted using a Genetic Microsystems array generator, and an Axon GenePix Scanner. Microarrays are prepared by first amplifying cDNA fragments encoding marker sequences to be analyzed, and spotted directly onto glass slides to compare mRNA preparations from two cells of interest, one preparation is converted into Cy3-labeled cDNA, while the other is converted into Cy5-labeled cDNA. The two cDNA preparations are hybridized simultaneously to the microarray slide, and then washed to eliminate non-specific binding. The slide is then scanned at wavelengths appropriate for each of the labels, the resulting fluorescence is quantified, and the results are formatted to give an indication of the relative abundance of mRNA for each marker on the array.

Alternatively, the PP1 gene product is determined by an antibody selected from the group consisting of a polyclonal antibody, a monoclonal antibody, a human antibody, humanized antibody, a chimeric antibody, and a synthetic antibody. Preferably, the antibody is detectably labeled or otherwise modified and/or to be detected by a secondary antibody.

Generally, methods for detecting PP1 expression or activity are well known to the person skilled in the art and can be found in such standard textbooks; see also supra and the appended examples. Reagents, detection means and kits for diagnostic purposes are available from commercial vendors such as Pharmacia Diagnostics, Amersham, BioRad, Stratagene, Invitrogen, and Sigma-Aldrich as well as from the sources given any one of the references cited herein, in particular patent literature. For example, international application WO2006/014645 describes generic probes that bind to phosphorylated amino acid residues as well as methods employing the probes for screening for kinase inhibitory activity, kinase activity, and phosphatase activity. Methods for distinguishing serine/threonine kinase phosphorylation from tyrosine kinase phosphorylation are also provided. In addition, international application WO2006/083016 and European patent application EP 1 199 370 describe means and methods for determining the activity of protein kinase and protein phosphatase, respectively, making use of peptide substrate and immunoassay techniques employing inter alia antibodies having a specificity to the substrate peptide or protein that is phosphorylated. The disclosure content of any one of those applications is incorporated herein by reference in their entirety, in particular with respect to the nucleic acid and antibody probes as well as reagents for detecting kinase and phosphatase activity for use in the diagnostic methods and kits therefore of the present invention.

Hence, the present invention also relates to a kit for use in any one of the above-described diagnostic methods, said kit comprising appropriate reagent means such as those described in the Protein Tyr Phosphatase (PTP) Assay System of New England Biolabs Inc. or the PP1/PP2A Toolbox of Upstate Inc., cell signaling solutions. In addition, or alternatively, the kit comprises an appropriate antibody and/or nucleic acid molecule, as mentioned before, which are specific for the phosphatase and encoding mRNA/cDNA, respectively. Suitable reagents include, but are not limited to, PP1 enzyme, PP2A enzyme, PHI-1 protein, okadaic acid, calyculin A, protein phosphatase dilution buffer, BSA, etc.

A further object of the present invention is to provide a kit and assay, see Example 7, adapted for a non-radioactive protein serine/threonine phosphatase activity assay, useful for e.g. multiple subcellular compartments from brain tissue such as those described in the examples. Typically, the kit of the present invention for the non-radioactive detection of the activity of PP1, PP2A, and calcineurin (PP2B), comprises:
(a) an inhibitor that preferentially inhibits either PP1 alone or PP2A alone;
(b) a compound capable of interfering with protein kinase A activity and blocking phosphorylation-dependent endogenous phosphatase inhibitors;
(c) a compound capable of blocking calcineurin activity; and optionally
(d) a phosphatase substrate.

The advantages of this kit and assay, respectively, are that it does not require any radioactive labeling but takes advantage of colorimetric detection of inorganic phosphates (BIOMOL© Green), and is capable of detecting and distinguishing the activity of PP1, PP2A, and calcineurin (PP2B). The kit of the present invention can be successfully used to detect phosphatase activity in subcellular fractions from fresh and frozen brain structures and in acute slices used for electrophysiological recordings such as those described in the examples.

The kit and assay of the present invention is based on the measurement of free phosphates released from a specific phosphatase substrate, an RII fragment derived from the catalytic subunit of the protein kinase PKA, by different serine/threonine phosphatases. The assay distinguishes the activity of the different protein phosphatases acting on this substrate by the use of specific inhibitors and reaction conditions. After reaction with the RII substrate, free phosphates are purified by TCA precipitation then quantified by reaction with the BIOMOL© Green reagent. Surprisingly, the reactions conditions referred to in the radioactive Protein Tyr Phosphatase (PTP) Assay System and kit of New England Biolabs (NEB), see for example Catalog# P0785S, proved successful for the non-radioactive assay of the present invention and for the detection of calcineurin activity as well. Thus, in one embodiment the kit and assay of the present invention, respectively, includes RII phosphopeptide substrate for example from the BIOMOL kit as well the BIOMOL Green reagent and the reaction conditions from the NEB kit. Accordingly, the present invention relates to a kit comprising means for a non-radioactive protein serine/threonine phosphatase activity assay, which is capable of detecting and distinguishing the activity of PP1, PP2A, and calcineurin (PP2B). The kit of the present invention typically has means include buffer or ingredients therefor comprising one or more of caffeine, calmodulin, tautomycin, okadaic acid, RII phosphopeptide and/or a colorimetric detection reagent; see also Example 7.

The kit of the present invention preferably includes the pharmacological inhibitors tautomycin (IC50: 1.1-7.5 nM for PP1 and 10-23 nM for PP2A) and okadaic acid (IC50: 10-1300 nM for PP1 and 0.02-1.0 nM for PP2A) at different concentrations to preferentially inhibit either PP1 alone or PP2A alone. To improve the detection of PP1 and PP2A activity, caffeine is further preferably added to the reaction buffer to interfere with protein kinase A activity and neutralize endogenous phosphorylation dependent phosphatase inhibitors. A high concentration of a Ca²⁺ chelator such as EDTA is also preferably added to block calcineurin activity.

In a further preferred embodiment the kit and assay of the present invention includes a PiBind resin or equivalent means for eliminating interfering free phosphates from both, samples and substrate to further improve the detection sensitivity of phosphatase activity in the samples. Preferably, the kit and assay of the present invention includes Trichloroacetic acid (TCA) to precipitate out all reactive proteins from the reaction and immediately terminate the reaction. In addition the kit of the present invention includes reagents suitable provide or adjust TCA at a solution of about 48% TCA and RII phosphopeptide at a concentration of about 0.75 mM.

In a still further preferred embodiment the kit and assay of the present invention the sample to be analyzed is a complex tissue rather than purified solutions or non-complex samples such as cultured cells since protein phosphatases are differentially distributed and regulated in distinct cellular compartments to govern various cellular processes. Advantageously, the kit of the present invention also includes a manual or instructions for use in accordance with the details given in Example 7, in particular with respect to the assay buffers to be preferably used.

Furthermore, developing a drug based on the agent capable of modulating protein phosphorylation has been proven useful in accordance with the present invention, including obtaining marketing authorization and actually putting the authorized drug on the market can be achieved by a different company. Thus, in a further aspect the present invention relates to a method of conducting a drug development business comprising licensing, to a third party, the rights for further drug development and/or sales for therapeutic agents identified or profiled in accordance with the present invention, or analogs thereof.

For suitable lead compounds that have been provided, further profiling of the agent or analogs thereof can be carried out for assessing efficacy and toxicity in animals, depending on the modalities of the agreement with the respective third party. Further development of those compounds for use in humans or for veterinary uses will then be conducted by the third party. The subject business method will usually involve either the sale or licensing of the rights to develop said compound but may also be conducted as a service, offered to drug developing companies for a fee.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. Several documents are cited throughout the text of this specification. Full bibliographic citations may be found at the end of the specification immediately preceding the claims. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### EXAMPLES

The examples which follow further illustrate the invention, but should not be construed to limit the scope of the invention in any way. Detailed descriptions of conventional methods, such as those employed herein can be found in the cited literature; see also “The Merck Manual of Diagnosis and Therapy” Seventeenth Ed. ed by Beers and Berkow (Merck & Co., Inc. 2003).

The practice of the present invention will employ, unless otherwise indicated, conventional techniques of cell biology, cell culture, molecular biology, transgenic biology, microbiology, recombinant DNA, and immunology, which are within the skill of the art.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11 (2001), 98-107.

### Supplementary Methods

### Animals

Adult C57B1/6J mice or I-1* mutant and control littermates (9 to 12 weeks) were used. I-1* mice were obtained as previously described (Genoux et al., 2002) and were backcrossed to the C57B16/J background for 8-10 generations. Mutant mice carry CaMKIIalpha promoter-rtTA and tetO-I-1* transgenes, and control mice are littermates carrying no transgene or either one of the transgenes. Mice were fed with 6 mg/g doxycycline (Westward Pharmaceuticals Corp.) mixed with wet food (50:50 food to water ratio) for at least 7 days prior to experiments. All experiments were performed in accordance with Swiss Federal Veterinary Office regulations and by experimenters blind to genotype.

### Electrophysiological Recordings

Adult mice were killed by cervical dislocation and the heads were immediately immersed in freshly prepared ice-cold artificial cerebrospinal fluid (aCSF: 119 mM NaCl, 11 mM D-glucose, 1.3 mM MgCl₂.6H₂O, 1.3 mM NaH₂PO₄, 2.5 mM KCl, 2.5 mM CaCl₂, 26 mM NaHCO₃, gassed with 95% O₂/5% CO₂ for a minimum of 20-min) for at least 3 min. Brains were quickly removed and hippocampi were extracted for electrophysiological recordings. Acute slices (400 μm thick) were prepared with a vibratome (Leica VT 1000S, Germany) in ice-cold oxygenated aCSF. Slices were transferred to an interface chamber at 34° C. for 40 to 50 min then kept at room temperature for at least 1-2 hours before recording. Recordings were performed in a submersion chamber (Slice Mini Chamber III/IV, Luigs & Neumann, Germany) continuously perfused with aCSF at approximately 1.1 ml/min. A monopolar electrode was placed in the stratum radiatum to activate Schaffer collaterals and test stimuli were applied at 0.033 Hz at an intensity set to evoke 1/3 of the maximum f-EPSP. Evoked f-EPSP were recorded in the stratum radiatum with a borosilicate micropipette filled with NaCl 3M or aCSF. The signal was amplified with an AXOPATCH 200B amplifier (Axon Instruments/Molecular Devices, Germany) and sampled using pCLAMP. Baseline responses were recorded for 10-min or until stable. For extracellular recordings in example 8, a monopolar electrode is placed in the Schaffer collateral fibers, and stimulation is applied at 0.033 Hz with stimulus intensity ranging from 20 to 80 μA, yielding evoked field EPSPs (fEPSPs) of 0.2-0.5 V. fEPSPs are recorded in the stratum radiatum using a borosilicate micropipette filled with aCSF.

### OGD

Acute slices (hippocampus, cortex and striatum) were exposed to 15 to 25-min of hypoxic/aglycemic conditions by perfusion with aCSF containing no glucose (replaced with equimolar sucrose) and gassed with N₂/CO₂ (95/5%) (Lobner and Lipton, 1993; Raley-Susman and Lipton, 1990). In example 8 Glucose in the aCSF was replaced with 8 mM sucrose and 3 mM 2-deoxyglucose. The OGD solution reached the recording chamber within 30 seconds and replaced the normoxic aCSF within 2-3 min.

### LTP/LTD

LTP was induced by a 1 sec train at 100-Hz, and LTD by a 10 min paired pulse stimulation at 2 Hz for with 200 ms inter-stimuli interval, applied to the Schaffer collaterals. In experiments combining LTP or LTD with OGD, normoxic aCSF was replaced with OGD aCSF either 10 or 30 min after the end of LTP or LTD induction. Two-pathway experiments were carried out by placing stimulating electrodes in stratum radiatum on either side of the recording electrode. Alternating test stimuli were given every 15 or 7.5 sec for LTP or LTD experiments, respectively. HFS or LFS were induced on alternating electrodes to ensure that both pathways could be modulated. Conditioning stimuli were delivered to both pathways in slices used for the phosphatase assay.

### Treatment and Drugs

Drugs for slice recordings were bath-applied for 2 hours before recording in oxygenated aCSF. Okadaic acid (Alexis Corporation, Switzerland) and tautomycin (Alexis Corporation and Sigma Aldrich, Switzerland) were dissolved in DMSO (30 mM) and methanol or ethanol (1 mM), respectively, and stock solutions were diluted further in aCSF the day of the experiment. The respective solvents were used for drug-control experiments. For both mutant and control slices, the aCSF was supplemented with doxycycline hydrochloride (Sigma, Buchs, Switzerland) at 8 ng/ml.

### Phosphatase Assay

Hippocampal slices were stimulated on two pathways with HFS or LFS 10 min prior to OGD as described above. CA1 was dissected either immediately (OGD 0 min) or 1 hr (OGD 1 hr) after OGD, homogenized in lysis buffer (3.75 mM Tris-HCl, pH7.4, 15 mM KCl, 3.75 mM NaCl, 250 μM EDTA, 50 μM EGTA, 30% (v/v) glycerol, 15 mM β-Mercaptoethanol, protease inhibitor cocktail (Sigma-Aldrich, Switzerland), 100 μM PMSF), and frozen on dry ice or in liquid nitrogen. Phosphatase activity was determined by incubating an approximately 2 μg sample with 0.15 mM RII substrate and 5 nM Tautomycin (for PP1 activity) or 5 nM Tautomycin/5 nM OA (for PP1 and PP2A activity) in buffer containing 50 mM Tris-HCl, pH 7.0, 100 μM Na₂EDTA, 5 mM DTT, and 0.01% Brij 35 at 30° C. for 10 min. The reaction was terminated by adding TCA and centrifuging at 13,000 g for 5 min. The amount of free phosphates released in the reaction was measured by mixing the supernatant with BIOMOL Green reagent (BIOMOL International LP, Switzerland) and detecting the color density at OD 620 nm. For determining total phosphatase activity, tautomycin and OA were removed from the reaction. Background activity was measured separately in a reaction without RII substrate and in a reaction without sample. The percent phosphatase activity was calculated by subtracting the background from the total and inhibited reactions, then dividing the inhibited activity by the total activity.

### Intraluminal MCAO

Adult I-1* mutant and control littermates (21-28 g) were anesthetized with isofluorane and subjected to focal cerebral ischemia by intraluminal MCAO using an 8-0 silicon coated (Xantopren, Bayer Dental, Osaka, Japan) nylon monofilament (Ethilon, Ethicon, Norderstedt, Germany). Laser Doppler flow (LDF) was monitored using a flexible 0.5 mm fiberoptic probe (Perimed, Stockholm, Sweden) attached to the intact skull overlying the MCA territory. Rectal temperature was maintained at 36.5-37.0° C. with a feedback-controlled heating system. After MCAO, wounds were carefully sutured and anaesthesia was discontinued. Following 24 hr (90 min MCAO) or 72 hr (30 min MCAO) of reperfusion, animals were anaesthetized with isofluorane and sacrificed. Brains were removed, and either frozen on dry ice and cut into 18 μm cryostat sections or dissected out bilaterally for Western blot analyses.

### Histology

Brain sections were fixed in 4% paraformaldehyde/0.1M PBS and infarct volumetry was performed after cresyl violet staining. Five sections from equidistant brain levels (2 mm apart) were evaluated for each animal. Borders between infarcted and non-infarcted tissue were outlined using an image analysis system (Image J) (Spudich et al., 2006; Wang et al., 2005). Other sections were stained by terminal transferase biotinylated-dUTP nick end labelling (TUNEL) using a kit (Roche, Switzerland). The number of DNA fragmented cells was quantified by a stereological method using uniform random sampling of an array with six regions of interest (250 μm×250 μm per area) separated by 250 μm and mean values were calculated to determine the number of apoptotic cells as previously described (Hermann et al., 2001).

### Western Blots

Tissue samples from the striatum ipsilateral and contralateral to MCAO or from acute forebrain slices (hippocampus, cortex and striatum) were homogenized in lysis buffer and centrifuged. Supernatants were resolved on SDS-PAGE and proteins were transferred onto PVDF membranes. Membranes were dried, incubated in blocking solution then in rabbit anti-ERK1/2 (9102; Cell Signaling), mouse anti-phospho-ERK1/2 (anti-phosphoThr and Tyr, M8159; Sigma), rabbit anti-JNK1/2 (JNK2, sc-572; Santa Cruz), rabbit anti-phospho-JNK1/2 (anti-Tyr185, 9251; Cell Signaling), rabbit anti-Bcl-X (610212; BD Biosciences) or rabbit anti-activated caspase-3 (CM1; BD Biosciences) antibody (1:500 in 0.1% Tween 20, 0.1 M TBS) (Kilic et al., 2005). Membranes were rinsed, incubated in peroxidase-coupled secondary antibody (1:2000 in 0.1% Tween 20, 0.1 M TBS), washed, immersed in enhanced chemoluminescence (ECL) solution then exposed to ECL-Hyperfilm (Amersham). The band intensity was determined by densitometry and corrected for protein loading using anti-β-actin antibody (A5316; Sigma) as an internal control.

### Slice Culture and Viral Infection:

Overexpression of PP1 in vitro using a lentivirus-based vector expressing human PP1 inducibly and reversibly: Hippocampal slices from 5- to 7-day postnatal mice are prepared and cultured using the roller-tube technique. Stocks of virus particles are diluted in culture medium supplemented with 10 mM MgCl2 and 0.5 μM tetrodotoxin (to reduce excitotoxic injury during infection). For virus injections, slices are transferred from the roller tubes in a chamber containing 2-3 ml of culture medium supplemented with 10 mM MgCl2 and 0.5 μM tetrodotoxin. Infections are performed at room temperature by microinjection of virus into the extracellular space of the slice cultures. Pipettes are filled with viral solution and lowered into the CA1 pyramidal cell layer by a micromanipulator. For each position, one injection of a few sec is performed by applying pressure from a 1-ml syringe. Typically, 3-10 injection sites are selected per slice, and a total of 2 μl of the virus solution is applied. After injection, slices are returned to the roller tubes, and cultured for at least 1 week.

### Data Analysis

For each electrophysiological experiment, the average slope of individual f-EPSP was measured from the initial 1-1.5 ms-portion following the fiber volley using Clampfit (Axon Instruments, Germany). For statistical analyses, the f-EPSP slope was averaged over 1-min and analyzed by three-way analysis of variance (ANOVA) using Statview 5.0 with sequences (10-min) and stimulation (1-min bins) as repeated measures. The between-subjects factors were OA dose (control versus 0.1, 1, 10 or 100 nM, or 1 genotype (controls versus mutants), delay between LTP or LTD and OGD (controls versus 10- or 30-min), genotype on LTD and LTD+OGD (controls versus mutants) and combined genotype-drug treatment on LTD and LTD+OGD (controls versus mutant+tautomycin). Unpaired t-tests were performed on mean f-EPSP for 10 min towards the end of recording as indicated in Results, when main effect of the between-subjects factor was significant. In some cases, the generalized linear model (GLM) followed by Tukey or LSD post-hoc tests were used to evaluate electrophysiological or phosphatase assays data. Infarct volume and DNA fragmentation were analyzed by two-tailed t-tests. Western blots were evaluated by one-way ANOVA followed by LSD post-hoc tests. Statistical significance was set at p=0.05, data are presented as mean±SEM.

### Example 1

### Pharmacological Inhibition of PP1 Reduces Recovery from OGD in Acute Hippocampal Slices

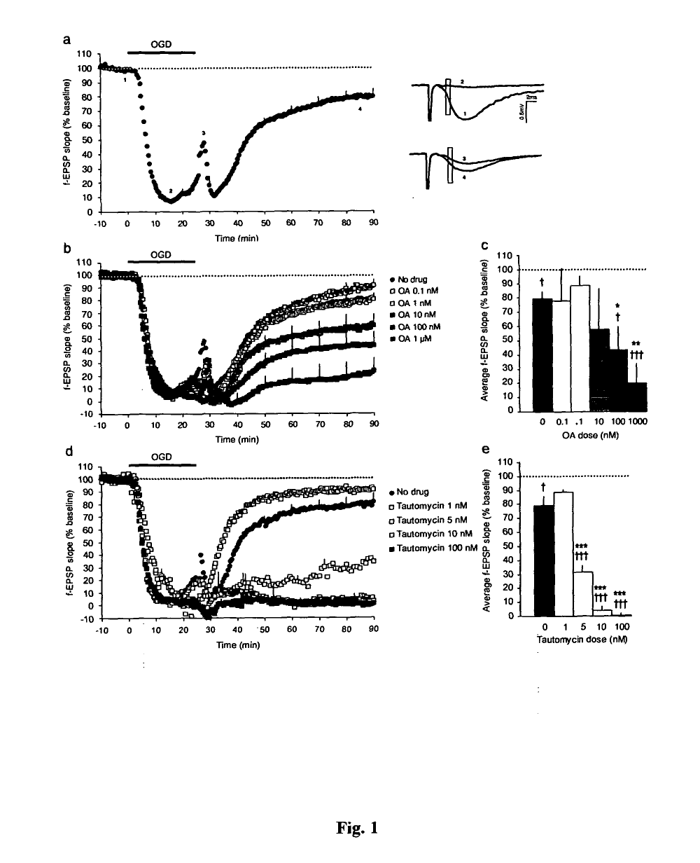

To model conditions of transient cerebral ischemia in vitro, acute hippocampal slices were subjected to 25 min OGD, and the effect of OGD was evaluated by recording evoked field excitatory postsynaptic potentials (f-EPSP) in area CA1 (Lobner and Lipton, 1993; Raley-Susman and Lipton, 1990). OGD induced a rapid decline in the slope and amplitude of the evoked f-EPSP followed by a partial recovery and stabilization of the f-EPSP to a level that was significantly lower than prior to OGD (f-EPSP slope: 79.2±4.9% of pre-OGD baseline 80-90 min after OGD onset, n=10, unpaired t-test: t₂₀=5.6, p<0.05, FIG. 1a). The involvement of PP1 in f-EPSP recovery after transient OGD was examined by testing the effect of okadaic acid (OA), a natural toxin that inhibits PP1 and PP2A with different efficacy (IC₅₀ for PP1: 20-100 nM; for PP2A: 0.1-1 nM (Cohen et al., 1990)). Graded doses of OA were used to inhibit both PP1 and PP2A, or PP2A alone. At doses sufficient to inhibit PP1 (10 nM-1 μM), OA significantly reduced f-EPSP recovery but had no effect at lower doses (0.1-1 nM) (mean f-EPSP slope 80-90 min after the onset of OGD: F_{(5, 29)}=3.54, p<0.05; 0.1 nM: 76.6±20.5%, n.s, n=4; 1 nM: 86.8±5.2%, n.s., n=2; 10 nM: 56.6±23.9%, n.s., n=4; 100 nM: 43.1±15.6%, p<0.05, n=8; 1 μM: 20.3±14.4%, p<0.001, n=8; versus 0 nM control slices (n=10) 79.2±4.9% p<0.005; FIGS. 1b, c), indicating that PP1 inhibition impairs f-EPSP recovery. To confirm that the effect was specific to PP1, the experiments were repeated using tautomycin, a more selective drug that inhibits PP1 with higher efficacy than PP2A (IC₅₀ for PP1: 0.1-1 nM; for PP2A: 10-20 nM (Gupta et al., 1997; MacKintosh and Klumpp, 1990). Consistent with the data using OA, tautomycin at a low dose (3 nM) significantly reduced f-EPSP recovery (mean f-EPSP slope between 80-90 min after the onset of OGD: F_{(5, 20)}=139.8, p<0.3; 1 nM: 88.6±1.5%, n.s., n=5; 3 nM: 31.4±4.6%, p<0.001, n=5; 10 nM: 4.4±2.9%, p<0.001, n=3; 100 nM: 0.9±1.3%, p<0.001, n=5; versus control slices (n=4) 79.3±6.2% p=0.01; FIGS. 1d, e) but a dose of 1 nM, not sufficient to inhibit enough of PP1 (about 30%) had no effect (FIG. 1d, e). Higher doses (10 and 100 nM) that fully inhibit PP1 but also inhibit PP2A however abolished recovery.

### Example 2

### Genetic Inhibition of PP1 Reduces f-EPSP Recovery after OGD

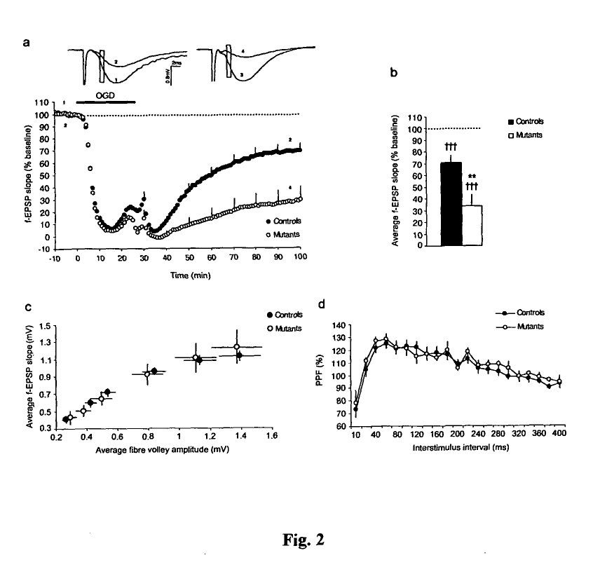

To confirm that PP1 inhibition is responsible for the reduced f-EPSP recovery after transient OGD, a genetic approach was used to specifically inhibit PP1 in the adult brain. Hippocampal slices from transgenic mice expressing a constitutively active form of the PP1 inhibitor, inhibitor-1 (I-1*) (Genoux et al., 2002) selectively in forebrain neurons were prepared and subjected to OGD. In these mice, I-1* expression in neuronal cells leads to partial inhibition of PP1 (67.7±12% (Genoux et al., 2002)) in the hippocampus. I-1*-mediated PP1 inhibition significantly reduced f-EPSP recovery such as f-EPSP slope recovered to only 34.2±9.7% of baseline 90 min after the onset of OGD compared to 71.3±5.8% in control slices (mean effect of genotype F_{(1, 28)}=11.81, p<0.005, Controls n=13, Mutants n=17, FIG. 2a, b). The degree of recovery in the mutant slices was comparable to that in control slices treated with 100 nM-1 μM OA (FIG. 1b, c) or 3 nM tautomycin (FIG. 1d, e), suggesting a specific effect of PP1 inhibition. This effect was not attributable to any gross change in basal properties of synaptic transmission resulting from transgene expression since the correlation between stimulation intensity and synaptic response (input-output) and paired-pulse facilitation, a short-lasting increase in synaptic efficacy mediated by presynaptic mechanisms, were normal (FIG. 2c, d). Overall, these results provide further evidence that inhibition of PP1 impairs recovery after transient OGD in hippocampal slices

### Example 3

### The Induction of LTP Prior to OGD Mimics the Effect of PP1 Inhibition

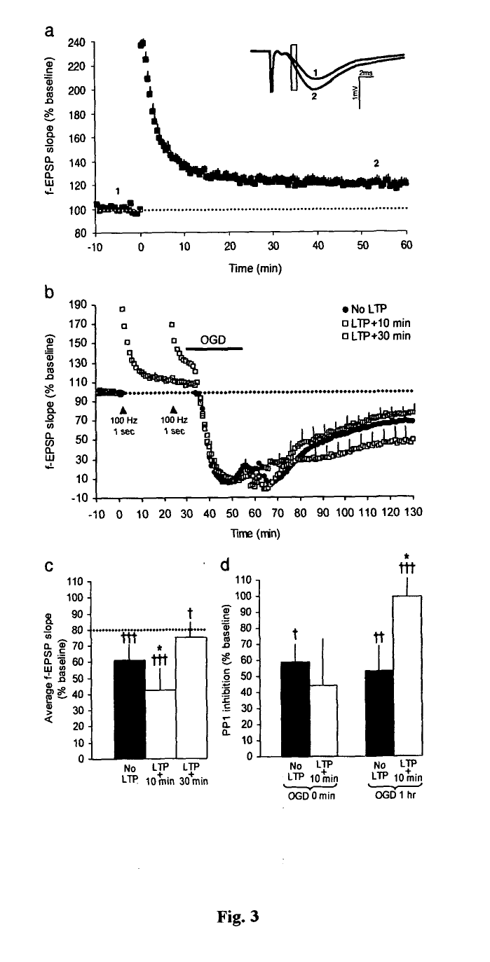

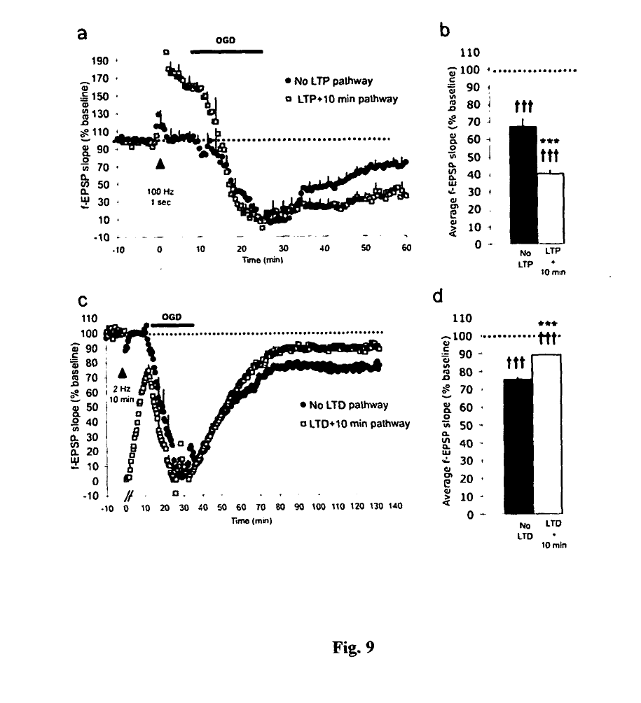

Signaling cascades involving the Ser/Thr protein phosphatases PP1, PP2A and CN are known to be modulated by synaptic plasticity. In particular, LTP and LTD, two major forms of synaptic plasticity in the hippocampus, are accompanied by opposite regulation of phosphatase activity. Both PP1 and PP2A were reported to be inhibited shortly after the induction of LTP but, while PP1 activity fully recovers after about 30 minutes, PP2A activity remains suppressed (Blitzer et al., 1998; Brown et al., 2000; Fukunaga et al., 2000; Morishita et al., 2001; Thiels et al., 1998; Winder and Sweatt, 2001). In contrast, CN is activated after the induction of LTP (Lu et al., 2000; Winder and Sweatt, 2001). Advantage of these features was taken to differentially modulate phosphatase activity by inducing LTP or LTD prior to OGD. First it was tested whether LTP can alter recovery by stimulating Schaffer collaterals with high frequency stimulation (HFS, 100 Hz, 1 sec) before OGD induction (FIG. 3a). When LTP was elicited 10 min prior to OGD (LTP+10 min), f-EPSP dramatically decreased then partially recovered but remained strongly depressed (31.6±9.9% of baseline 90 min after OGD onset, n=11). In contrast, when LTP was induced 30 min prior to OGD (LTP+30 min), f-EPSP recovery was similar to recovery in slices subjected to OGD alone (No LTP) (mean f-EPSP slope 80-90 min after OGD onset: F_{(4,62)}=7.03, p<0.001; No LTP: 69.3±3.5%, n=33 versus LTP+10 min, 40.6±13.7%, n=11, Tukey post-hoc <0.005; versus LTP+30 min, 72.0±8.0%, n=6, Tukey post-hoc n.s; FIGS. 3b, c), indicating no effect of LTP+30 min. To confirm that the detrimental effect of LTP+10 min was specific to the stimulated pathway, 2-pathway recordings were performed in slices subjected to OGD and applied HFS to only one pathway 10 min prior to OGD. A significant decrease in the f-EPSP slope recovery was observed selectively in the stimulated pathway (LTP+10 min pathway) while recovery in the non-stimulated pathway (No LTP) was comparable to that with OGD alone (No LTP, FIG. 3b, c) (F_{(2,6)}=193.5, p<0.001, No LTP pathway: 70.0±3.5%, n=4; LTP+10 min pathway: 37.5±3.5%, n=4, Tukey post-hoc p<0.001, FIG. 9), confirming the selectivity of the effect of HFS. Notably, the level of f-EPSP recovery in slices subjected to LTP+10 min, whether in one or 2-pathway recordings, was comparable to that observed following pharmacological or genetic inhibition of PP1 (% of pre-ischemic level: 43.5±16.6% for 100 nM OA, 20.2±13.9% for 1 μM OA, 31.4±4.6 for 3 nM tautomycin, 34.2±9.7% for I-1*, t₁₇=0.55, p=0.59, FIGS. 1 and 2), further supporting the hypothesis that PP1 inhibition is responsible for the reduced OGD recovery.

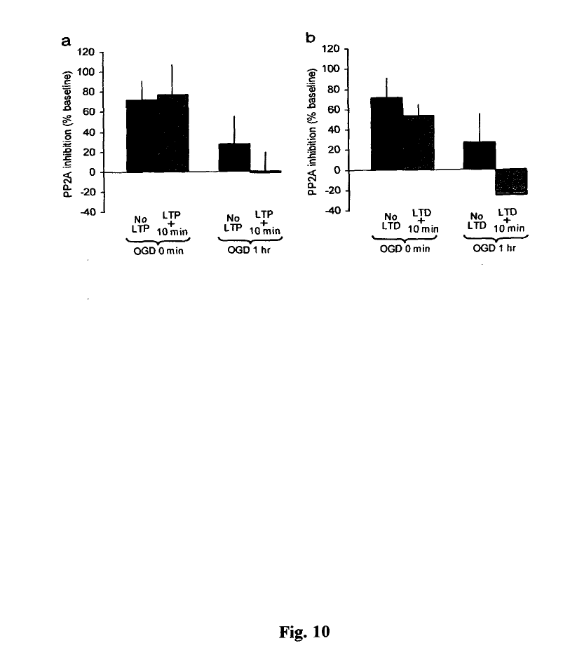

To provide biochemical support to these findings, PP1 activity was measured immediately (OGD 0 min) or 1 hour (OGD 1 hr) after OGD in hippocampal slices subjected to LTP+10 min, and compared it to the activity in non-stimulated (no LTP) OGD slices. OGD alone was found to induce a rapid and significant inhibition of PP1 (OGD 0 min: F_{(3,44)}=2.947, p<0.05; No LTP: 56.4±12.4% inhibition from baseline, n=10, LSD post-hoc p<0.05) that persisted for at least 1 hour (OGD 1 hr: F_{(3,38)}=14.98, p<0.001; No LTP: 52.9±15.9% inhibition from baseline, n=8, Tukey post-hoc p<0.05, FIG. 3d). Remarkably however, PP1 inhibition was even more pronounced 1 hour after OGD when LTP was induced 10 min prior to OGD (LTP+10 min at OGD 1 hr: 99.5±17.0% inhibition from baseline, n=9, Tukey post-hoc p<0.001 compared to baseline, LSD post-hoc p<0.05 compared to No LTP OGD 1 hr), indicating that LTP induction exacerbates the OGD-mediated reduction in PP1 activity. Importantly, this effect of LTP was specific to PP1 and did not involve PP2A. Although PP2A activity was also partially inhibited immediately after OGD, its activity was not changed by prior LTP (OGD 0 min: No LTP, 71.4±2.2%, n=7; LTP+10 min, 76.8±30%, n=6). PP2A activity was further only transiently inhibited and was fully restored 1 hour after OGD whether LTP was induced or not (OGD 1 hr: No LTP, 27.2±27.8%, n=7; LTP+10 min, −1.6±20.8%, FIG. 10). Altogether, these results strongly suggest that LTP impairs recovery from OGD through mechanisms involving PP1 inhibition.

### Example 4

### Prior Induction of LTD Promotes Recovery after OGD

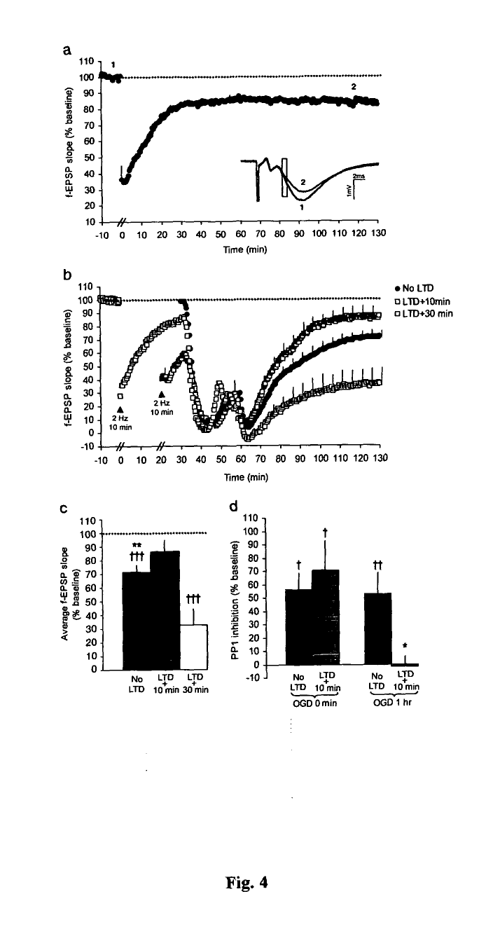

To test whether synaptic plasticity has a general impact on OGD recovery, it was next examined if LTD, a long-lasting weakening in synaptic efficacy, may have an opposite effect to LTP. Similar to LTP-OGD experiments, LTD was evoked 10 min (LTD+10 min) or 30 min (LTD+30 min) prior to OGD by Schaffer collaterals stimulation (low frequency stimulation (LFS), 10 min at 2 Hz, paired pulse). It was observed that contrary to LTP+10 in, LTD+10 min induced a full recovery of f-EPSP. While f-EPSP initially diminished after OGD, they rapidly recovered and reached a level comparable to that in slices subjected to LTD alone without OGD (LTD alone: 84.7±1.4% of baseline 80-90 min after LFS, n=6; LTD+10 min before OGD: 85.6±7.7% of baseline 80-90 min after OGD onset, n=6, FIG. 4a, b). However, when LTD was induced 30 min prior to OGD, f-EPSP did not properly recover and were even lower than after r OGD alone or LTD alone (F_{(4,62)}=7.03, p<0.001; LTD+30 min: 29.3±11.2% of baseline 80-90 min after OGD onset, n=8, Tukey post-hoc p<0.005; FIG. 4a, b), maybe due to sustained activation of CN or PP2A, both of which known to be detrimental to cell survival (Fukunaga et al., 2000; Thiels et al., 1998; Winder and Sweatt, 2001). It was confirmed that the effect of LTD+10 min was specific to the stimulated pathway by performing two-pathway recordings. Consistent with a direct and selective effect of LFS, a significant increase in f-EPSP recovery was observed selectively in the stimulated pathway (LTD+10 min pathway) (89.2±1.0% of baseline 120 min after OGD, n=4), while recovery in the non-stimulated pathway (No LTD) was comparable to that in control OGD in one-pathway experiment (FIG. 4b) (75.4±0.5% of baseline 110-120 min after OGD, n=4) (FIG. 9b).

To confirm that the mechanisms of recovery induced by LTD involve PP1, PP1 activity was measured in hippocampal slices subjected to LTD+10 min and OGD, both immediately and 1 hour after OGD. PP1 activity was again found to be significantly inhibited immediately after OGD whether LTD was induced prior to OGD or not (OGD 0 min: baseline versus No LTD, 56.4±12.4% inhibition from baseline, n=10, LSD post-hoc p<0.05; versus LTD+10 min, 70.5±22.5% inhibition from baseline, n=6; LSD post-hoc p<0.05). However contrary to LTP, PP1 activity was found to be fully restored 1 hour after OGD with LTD+10 min (OGD 1 hr: baseline versus LTD 10 min, −2.0±8.1% inhibition from baseline, n=5; Tukey post-hoc p=1.0) whereas inhibition persisted after OGD in the absence of LTD (OGD 1 hr: baseline versus No LTD, 52.9±15.9% inhibition from baseline, n=8; Tukey post-hoc p<0.05; No LTD versus LTD+10 min, LSD post-hoc p<0.05, FIG. 4d). Similar to the results observed for LTP+10 min/OGD, the effect of LTD was specific to PP1 and did not involve PP2A (OGD 0 min: No LTD, 71.4±19.3%, n=7; LTD+10 min, 53.5±10.8%, n=5; OGD 1 hr: LTD alone, 27.2±27.8, n=7; LTD+10 min, −25.4±22.2%, FIG. 10b). These results overall clearly indicate that LTD induced shortly before OGD promotes the recovery of synaptic responses and that this effect involves the reversal of PP1 inhibition induced by OGD.

### Example 5

### The Beneficial Effect of LTD is Blocked by PP1 Inhibition

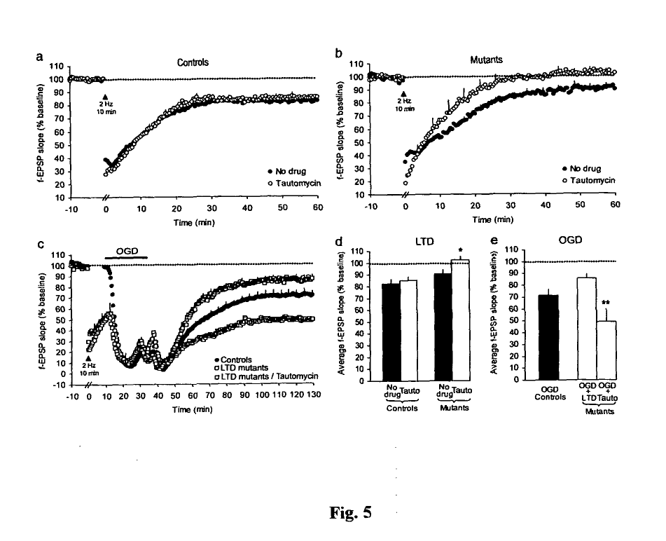

To confirm the involvement of PP1 in the beneficial effect of LTD, it was tested whether inhibiting PP1 by transgenic I-1* expression can abolish this effect in I-1* mutant hippocampal slices. When elicited 10 min prior to OGD, LTD induced a full recovery of f-EPSP in mutant slices (LTD alone, 87.6±4.6% of baseline at 90 min, n=7, versus control slices 83.2±1.7%, n=6, unpaired t-test: t₍₁₁₎=0.65, n.s., FIG. 5a, b, d, No drug; LTD+10 min, 85.4±3.8%, n=6 versus LTD alone in mutant slices, 87.6±4.6%, n=7 after 90 min, unpaired t-test: t₍₁₁₎=0.364, n.s., FIG. 4a, 5b-e). This unexpected recovery may be explained by the fact that PP1 inhibition is only partial in I-1* slices (67.7±12% (Genoux et al., 2002)), suggesting that residual PP1 activity may be sufficient to maintain the beneficial effect of LTD. To test this hypothesis, residual PP1 activity was inhibited using a submaximal dose of tautomycin (1 nM) known to inhibit PP1 partially (about 30%). This additional inhibition combined with I-1* expression abolished the beneficial effect of LTD on f-EPSP recovery in mutant slices (LTD: 96.3±3.6% of baseline at 90 min, versus controls, unpaired t-test: t₍₁₀₎=3.12, p<0.05, n=6 for both groups, FIG. 5b, d, Tautomycin; LTD+OGD: 37.0±10.8%, versus no drug: 85.4±3.8% of baseline 90 min after LTD induction, unpaired t-test: t₍₁₀₎=4.24, p<0.005, n=6 for both groups, FIG. 5c, e), but had no effect in control slices (LTD: 85.2±3.0% of baseline at 90 min, n=6, versus no drug, 82.6±3.0%, n=8, of baseline 90 min after LTD induction, F_{(1, 12)}=0.18, p=0.68, FIG. 5a, d, data not shown for LTD+OGD). These results provide strong evidence that the beneficial effect of LTD on OGD recovery involves PP1-dependent mechanisms.

### Example 6

### Genetic Inhibition of PP1 Aggravates Infarct after Focal Cerebral Ischemia In Vivo

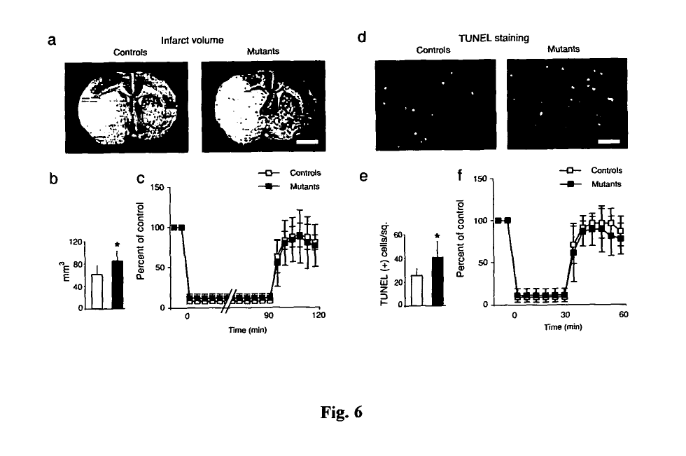

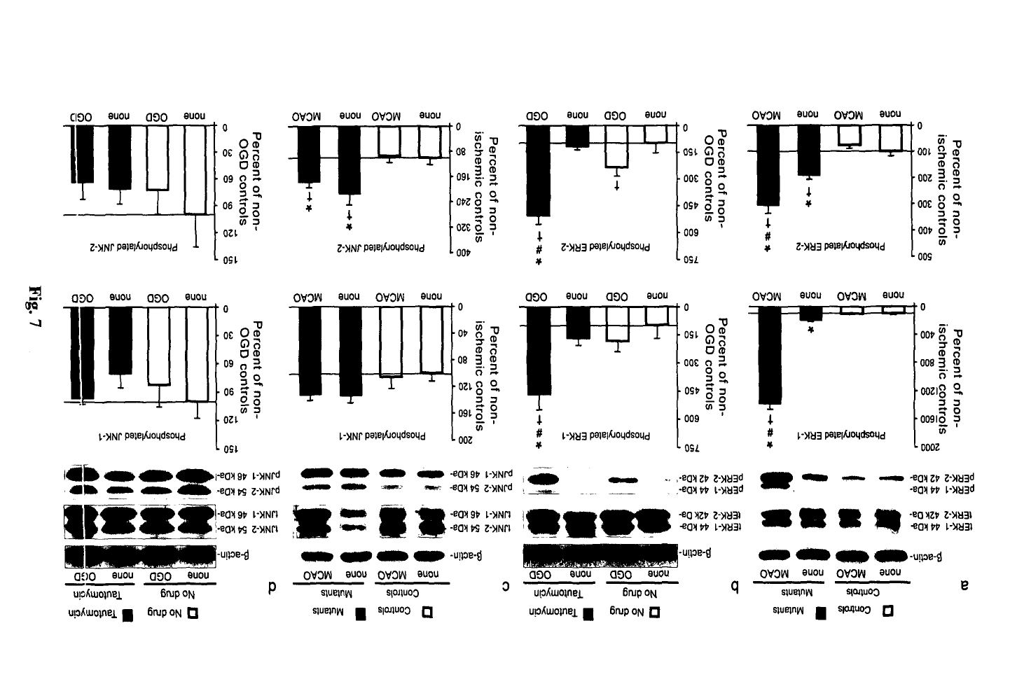

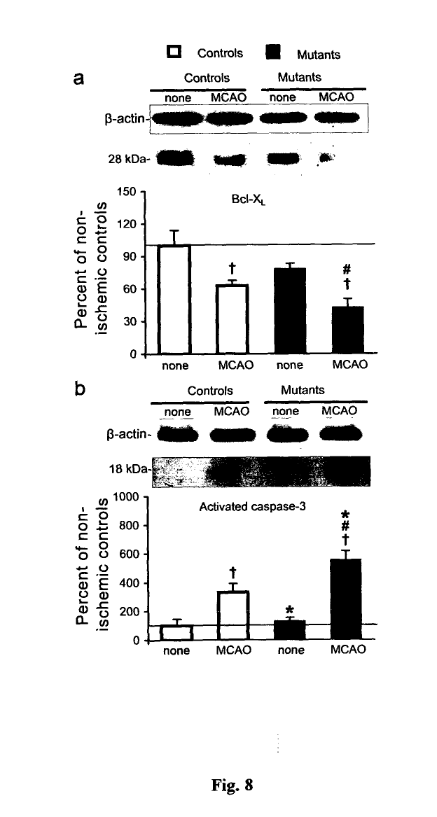

To validate the present findings in vivo, it was next examined whether the genetic inhibition of PP1 in adult mice affects the extent of damage after ischemia. I-1* mice and control littermates were subjected to intraluminal MCAO for 30 or 90 min and the effect was evaluated on brain tissue 24 or 72 hours later, respectively. Consistent with the present results in vitro, PP1 inhibition significantly enlarged the infarct volume in I-1* mutant mice compared to control mice (two-tailed t-test, p<0.05, n=8/group, FIG. 6a, c). It also induced disseminate cell death as indicated by increased TUNEL staining in I-1* mutant mice compared to control littermates (two-tailed t-test, p<0.05, n=6/group, FIGS. 6b, d). The aggravation of brain injury was not due to a difference in haemodynamic changes during or after MCAO since laser doppler flow (LDF) measurements were similar in I-1* mutant and control mice (FIG. 6e, f). To investigate the intracellular signaling pathways potentially involved in the effect of PP1 inhibition, it was examined whether selected targets associated with apoptotic/survival pathways were altered. Western blot analyses revealed that PP1 inhibition significantly increased the phosphorylation level of ERK1 (+98.1±22.3%; ANOVA/LSD-post-hoc, p<0.05, n=3/group), ERK2 (+91.0±17.8%; ANOVA/LSD-post-hoc, p<0.05, n=3/group) and JNK2 (+114.1±35.3%; ANOVA/LSD-post-hoc, p<0.05, n=3/group) but not JNK1 (+33.4±10.8%; ANOVA/LSD-post-hoc, n.s., n=3/group) (FIG. 7a, c). Strikingly, MCAO in I-1* mutant mice dramatically increased the level of phosphorylated ERK1 (+599.0±42.2%; ANOVA/LSD-post-hoc, p<0.05, n=3/group) and ERK2 (+60.1±16.0%; ANOVA/LSD-post-hoc, p<0.05, n=3/group) (but not JNK1 or 2) (FIG. 7a, c), suggesting that ERK1/2 activation is involved in the effect of PP1 inhibition. Consistently, a comparable effect on ERK1/2 was observed in vitro. Phosphorylated ERK1/2 were significantly increased in control slices treated with tautomycin and subjected to OGD (ERK1: +366.8±48.6%; ANOVA/LSD-test, p<0.05, n=3; ERK2, +404.1±28.5%; ANOVA/LSD-post-hoc, p<0.05, n=3, FIG. 7b), providing strong evidence for a link between PP1 inhibition and ERK1/2 phosphorylation. Also similar to that observed in vivo, JNK1 phosphorylation was not altered by PP1 inhibition or OGD in vitro, however unlike in vivo, PP1 inhibition did not increase phosphorylated JNK2 in vitro (FIG. 7d), maybe due to the different duration of PP1 inhibition in vitro and in vivo (a few hours for tautomycin treatment in vitro versus several days of I-1* expression in the mutant mice) or to different JNK2 phosphorylation stoichiometry in vitro and in vivo. In addition to increasing ERK1/2 and JNK2 phosphorylation, the genetic inhibition of PP1 in vivo also accentuated the decrease in expression of the anti-apoptotic protein Bcl-X_{L} induced by MCAO (−49.0±13.5%; ANOVA/LSD-post-hoc, p<0.05, n=3/group, FIG. 8a) and the increase in activated caspase-3 (+60.2±19.7%; ANOVA/LSD-post-hoc, p<0.05, n=3/group, FIG. 8b), indicating that several pathways associated with apoptosis and cell survival are involved in the effect of PP1 inhibition.

The Significance of the Results of Examples 1 to 6 for the Treatment of Cerebral Neurological Disorder in Accordance with the Present Invention

The present results provide evidence that the serine/threonine protein phosphatase PP1 and PP1-dependent bidirectional plasticity are critical for the mechanisms of recovery from excitotoxicity in the adult mouse brain. The results demonstrate that a decrease in PP1 activity induced by pharmacological or genetic inhibition prior to OGD in hippocampal slices or prior to MCAO in vivo, alters the mechanisms of recovery. Furthermore in vitro, f-EPSP recovery is impaired when LTP is induced prior to OGD, an effect that is associated with the selective inhibition of PP1. In contrast, the induction of LTD prior to OGD promotes full f-EPSP recovery and is associated with restoration of PP1 activity after OGD. Consistently, the beneficial effect of LTD is abolished by blockade of PP1 activity, confirming that it involves PP1-dependent pathways. Several targets of cell death and cell survival pathways appear to be involved in the detrimental effect of PP1 inhibition since it is accompanied by increased phosphorylation of ERK1 and ERK2 both in vivo and in vitro, and increased JNK2 phosphorylation, caspase-3 activation, and decreased Bcl-X_{L} expression in vivo. Together, these findings reveal a novel molecular mechanism for brain recovery that fully relies on PP1, and they highlight yet unknown functions for LTP and LTD in the regulation of the brain response to injury.

Protein serine/threonine phosphatases have been suggested to promote neuroprotection (Fernandez et al., 1993; Klumpp and Krieglstein, 2002; Nuydens et al., 1998; Runden et al., 1998; Stevens et al., 2003; Yi et al., 2005) but the specific involvement of PP1 could not be examined until now due to the limited specificity of pharmacological inhibitors and the lack of selective activators of PP1. The only finding specific for PP1 after ischemia showed rather contradictory results revealing increased PP1α and PP1γ mRNA in the hippocampal CA1 region and the striatum, but decreased PP1γ protein with no change in PP1α (Horiguchi et al., 2002). In the present study, the combination of pharmacological, genetic and physiological methods allowed to specifically manipulate PP1 activity prior to experimental ischemia both in vitro and in vivo, and selectively demonstrate that PP1 is involved in the mechanisms of recovery from excitotoxicity. The manipulation further provided new evidence that ERK1 and 2 are under the control PP1 during an ischemic insult both in vitro and in vivo.

Many reports in models of cerebral ischemia, brain trauma or neurodegenerative diseases have demonstrated a detrimental role for ERK1/2 and JNK1/2 signaling (Bogoyevitch et al., 2004; Chu et al., 2004; Irving and Bamford, 2002). However, ERK1/2 can also be neuroprotective, most likely depending on the nature, duration and severity of injury, cell type and brain area, or timing of analysis (Hetman and Gozdz, 2004). The level of phosphorylated, activated ERK1/2 and JNKs was reported to rapidly increase in the post-ischemic brain (Farrokhnia et al., 2005; Shackelford and Yeh, 2006), and inhibition of ERK1/2 or JNKs suppresses neuronal injury following transient or permanent MCAO in the rat (Alessandrini et al., 1999; Borsello et al., 2003). The actual pathways linking the initial steps of excitotoxicity to ERK/JNK activation and apoptotic processes however remain not fully understood.

The present results now provide evidence that PP1 is a main regulator of ERK1/2 and JNK2 pathways, and a critical determinant of the response of neuronal cells to excitotoxicity that engage these pathways both in vitro and in vivo. This central function of PP1 suggests that it is strategically positioned upstream of major cell death/survival pathways, consistent with its ability to be retained near NMDA receptors at the membrane through scaffolding proteins (Westphal et al., 1999). There, PP1 may couple the NMDA receptor to downstream ERKs, JNKs and apoptotic pathways as suggested by the present correlated data between PP1 activity, ERK1/2 phosphorylation and recovery both in vitro and in vivo. It may dephosphorylate ERK1/2 directly (Quevedo et al., 2003) or indirectly by down-regulating upstream MEKs such as reported in rat pituitary cells (Manfroid et al., 2001). Further, the simultaneous increase in ERK1/2 and JNK2 phosphorylation induced by PP1 inhibition in vivo suggests that PP1 may in addition act to coordinate ERK1/2 and JNK2 pathways. This highlights a yet unknown PP1-dependent crosstalk between MAPK and JNK pathways that appears to operate a few hours after injury (not observed 1 hour after OGD in vitro). The ability of PP1 to associate with JNK2 in a tripartite complex in mitochondria may underlie this feature, and provide a means to tightly control this interaction (Brichese et al., 2004). Moreover, the concomitant activation of ERK1/2 and JNK2 may exacerbate the impact of excitotoxicity by activating the protease caspase-3. JNK1/2 activation is known to induce the translocation of the proapoptotic protein Bax to mitochondria. This operates by Bax release from cytoplasmic sequestration and cleavage of procaspase-3 into activated caspase-3 (Guan et al., 2006). Caspase 3-mediated apoptosis was indeed reported to be induced by PP1/PP2A inhibitors in several cell types in culture (Fladmark et al., 1999). In turn, activated caspase-3 can cleave anti-apoptotic proteins such as Bcl-X_{L} and Bcl-2, both of which known to associate with PP1 through the RVXF PP1-binding motif (Ayllon et al., 2000). Thus overall, the simultaneous upregulation of apoptotic proteins and downregulation of anti-apoptotic molecules induced by PP1 inhibition in vivo indicate that PP1 acts on multiple targets and pathways to limit excitotoxicity. The consistency between the present in vitro and in vivo data also strongly suggests that the mechanisms of recovery in these two systems share dependence on PP1 and ERK1/2, however it does not rule out the possibility that additional mechanisms are also recruited.

The present findings that two major forms of synaptic plasticity, LTP and LTD, modulate the mechanisms of OGD recovery reveal a novel physiological function for these bidirectional forms of plasticity in brain repair. The detrimental effect of LTP and the beneficial effect of LTD on recovery, and the correlated increased and decreased PP1 inhibition provide clear evidence that LTP and LTD oppositely module recovery both through PP1-dependent pathways. In addition to revealing a novel function for LTP and LTD, these results highlight a pivotal role for PP1 in the control of these forms of plasticity. This significantly extends previous findings showing that PP1 negatively regulates synaptic strength and plasticity (Jouvenceau et al., 2006; Morishita et al., 2001) by newly demonstrating that the control of LTP and LTD by PP1 has an important physiological relevance. The opposite effect of PP1 may here too derive from its strategic position, in particular in the control of the balance between protein kinases and phosphatases that regulate NMDA receptor-dependent signaling pathways (Mansuy and Shenolikar, 2006). A change in the activity of PP1 tilting this balance either in favor of kinases or phosphatases may explain the differential activation of cell survival or cell death pathways (Colbran, 2004; Lisman and Zhabotinsky, 2001).

The demonstration that LTP or LTD prior to OGD is detrimental or beneficial to recovery suggests that prior activity at synapses has a strong influence on the response to injury. This finding underscores the critical importance of the state of plasticity of synaptic circuits at the time of insult, a notion reminiscent of metaplasticity. Notably, a form of synaptic depression was previously suggested to enhance survival after transient OGD in a population of hippocampal CA1 neurons in vivo (Gao et al., 1998; Papas et al., 1993). Furthermore, in a similar preparation, neurons more likely to die were found to exhibit a form of potentiation that may be similar to anoxic LTP reported in vitro after short periods of OGD and that may act as a prelude to injury (Calabresi et al., 2003; Hori and Carpenter, 1994). These findings corroborate the present demonstration that LTD is neuroprotective and LTP neurotoxic. However, it should be noted that LTP was also suggested to confer protection during pre-conditioning when induced in between short episodes of OGD, through mechanisms involving Al adenosine receptors (Youssef et al., 2001; Youssef et al., 2003). However in these studies, brief OGD was also found to decrease synaptic responses (Youssef et al., 2001) and downregulate Ca²⁺ signaling, CaMKII and PKC (Katsura et al., 2001), which interfered with LTP induction, suggesting that neuroprotection may not have derived from LTP itself (Youssef et al., 2006). Finally, attenuation of kainic acid-induced neuronal loss by LTP was also reported, suggesting that LTP may have neuroprotective properties in certain conditions (YT Wang, SFN abstract 2005).

Lastly, the present study significantly advances understanding of the mechanisms of damage and protection in the context of ischemia and provides perspectives for the potential development of future treatment against brain pathologies such as ischemic stroke, traumatic brain injury or neurodegenerative disorders.

### Example 7

### Non-Radioactive Protein Serine/Threonine Phosphatase Activity Assay Useful for Multiple Subcellular Compartments from Brain Tissue

Protein phosphorylation is one of the key steps that orchestrate cellular processes. To study the importance of protein phosphorylation in cellular functions, it is essential to have appropriate methods to determine the activity of protein kinases and phosphatases in various tissues. For determining the specifically PP1 activity in the brain a reliable and efficient non-radioactive protocol was needed The specifics of such method are that it should not require any radioactive labeling and is able to optimally detect and distinguish the activity of PP1, PP2A, and calcineurin (PP2B). In the course of performing the experiments within the scope of the present invention a novel kit and assay system has been developed with increased specificity for PP1, PP2A or calcineurin (PP2B), which has been successfully used to detect phosphatase activity in subcellular fractions from fresh and frozen brain structures and in acute slices used for electrophysiological recordings.

The inclusion of a method for sample preparation from fresh or frozen brain tissue according to Genoux et al., Nature 418 (2002), 970-975 is a crucial addition to the protocol that allows the measurement of phosphatase activity in brain tissue. The commercially available kits are not designed for complex samples and are developed only for purified solutions or non-complex samples such as cultured cells. The present sample preparation is optimized to work with complex tissues, in particular brain, and is designed to obtain high quality samples for phosphatase assays. This is a key to obtain reproducible results, in particular for PP1 activity, which is notoriously variable. No sample preparation is included in the NEB kit and the preparation described in the BIOMOL kit is only appropriate for calcineurin activity but not for PP1 or PP2A. This latter preparation greatly differs from that of the present invention and is different for the lysis buffer components, homogenization steps, and final removal of free phosphates from the samples.

### Reagents

Samples for analysis or brain structure of interest, preferably 10 to 12 μg are used for PP1, PP2A and CN activity detection can essentially be prepared according to Genoux et al., Nature 418 (2002), 970-975. Protocols for nuclear extraction and synaptosomal lysate preparation in order to provide complex tissue samples are known in the art and can be based on the nuclear extraction method used in Dosemeci et al., Biochem. Biophys. Res. Commun. 339 (2006), 687-694 and Sarkar et al., Neuroscience 137 (2006), 125-132, whilst the synaptosomal preparation can be based on Hajos, Brain Res. 93 (1975), 485-489 and Dosemeci et al (2006), supra. Most preferably, the lysis and resuspension buffer recited below are used for crude preparation from brain tissue and brain slice homogenate preparation, respectively.

### Lysis Buffer:

| | | |
|---|---|---|
| Final Concentration | Volume | Stock to use |

| | | | |
|---|---|---|---|
| 10 mM HEPES, pH 7.4 | 5 | ml | 100 mM |
| 1 mM MgCl₂ | 0.5 | ml | 100 mM |
| 0.5 mM | 0.5 | ml | 50 mM |
| 250 μM EDTA | 125 | μl | 100 mM |
| 100 μM EGTA | 50 | μl | 100 mM |
| 0.32 M Sucrose | 5.47 | g | — |

| | | |
|---|---|---|
| Autoclaved MilliQ H₂O | Bring up to 50 ml | — |

### Resuspension Buffer:

| | | |
|---|---|---|
| Final Concentration | Volume | Stock to use |

| | | | | |
|---|---|---|---|---|
| 3.75 mM Tris-HCl, pH 7.4 | 187.5 | μl | 1 | M |
| 15 mM KCl | 750 | μl | 1 | M |
| 3.75 mM NaCl | 187.5 | μl | 1 | M |
| 250 μM EDTA | 125 | μl | 100 | mM |
| 100 μM EGTA | 50 | μl | 100 | mM |

| | | | |
|---|---|---|---|
| 30% glycerol | 15 | ml | — |
| 15 mM beta-mercaptoethanol | 52.45 | μl | — |
| 0.1% Brij 35 | 5 | ml | 1% |

| | | |
|---|---|---|
| Autoclaved MilliQ H₂O | Bring up to 50 ml | — |

Right before use, protease inhibitor cocktail and 100 μM PMSF (Sigma; 500× dilution of 50 mM PMSF in DMSO) (Sigma; 200× dilution) are added to the lysis buffer and resuspension buffer.

PiBind™ resin (Innova Biosciences Ltd)

### BIOMOL Green™ Reagent (BIOMOL International, LP)

### 25 μM Calmodulin (CaM)

48% Trichloroacetic acid (TCA; Sigma)
0.75 mM RII phosphopeptide (BIOMOL International, LP)

### 300 μM Tautomycin (Sigma) in 100% Ethanol

### 5 μM Okadaic Acid (OA; BIOMOL International, LP) in 100% Ethanol

96-well EIA/RIA plate(s), non-sterile (Corning Incorporated/Costar)

### Equipment

0.5 ml microcentrifuge tubes (labeled appropriately)
a plate reader with 620 nm filter (Multiskan Ascent; Thermo Labsystems)
a heating block, preheat to 30° C.
a microcentrifuge for 0.5 ml tubes, refrigerated (5810R; Eppendorf)
an incubation chamber (Mini10; MWG-Biotech), preheat to 30° C.; only needed if using a 96-well plate for the reaction, preheat to 30° C.
a centrifuge with a plate adaptor (5810R; Eppendorf); precool to 4° C.; only needed if using a 96-well plate for the reaction

### Reagent Setup

1× Assay Buffer 1 (no Calcium), of which approximately 200 μl are used per sample for PP1 and PP2A activity detection:

| | | |
|---|---|---|
| Final Concentration | Volume | Stock to use |

| 50 mM Tris-HCl, pH 7.0 | 150 | μl | 1 | M |
|---|---|---|---|---|
| 5 mM DTT | 30 | μl | 500 | mM |
| 0.01% Brij 35 | 30 | μl | 1% | |
| 1 mM Na₂EDTA | 30 | μl | 100 | mM |
| 1 mM MnCl₂ | 300 | μl | 10 | mM |
| 5 mM Caffeine | 300 | μl | 50 | mM |

| | | | |
|---|---|---|---|
| autoclaved MilliQ H₂O | 2160 | μl | — |

1× Assay Buffer 2 (with calcium for CN activity detection), of which approximately 50 μl per sample is used for CN activity detection:

| | | |
|---|---|---|
| Final Concentration | Volume | Stock to use |

| 50 mM Tris-HCl, pH 7.0 | 50 | μl | 1 | M |
|---|---|---|---|---|
| 5 mM DTT | 10 | μl | 500 | mM |
| 0.01% Brij 35 | 10 | μl | 1% | |
| 1 mM CaCl₂ | 10 | μl | 100 | mM |
| 1 mM MnCl₂ | 100 | μl | 10 | mM |
| 5 mM Caffeine | 100 | μl | 50 | mM |
| 50 nM Calmodulin | 2 | μl | 25 | μM |

| | | | |
|---|---|---|---|
| autoclaved MilliQ H₂O | 718 | μl | — |

### 5 nM Tautomycin:

| Final Concentration | Volume | Stock to use |
|---|---|---|
| 1x Assay Buffer 1 | 29.95 μl | 1x |
| 3 nM Tautomycin¹⁵ | 0.05 μl | 3 μM |

### 16.7 nM Okadaic Acid (OA):

| Final Concentration | Volume | Stock to use |
|---|---|---|
| 1x Assay Buffer 1 | 29.85 μl | 1x |
| 10 nM OA^{16, 17} | 0.1 μl | 5 μM |
| 3 nM Tautomycin¹⁵ | 0.05 μl | 3 μM |
| (optional) | | |

48% TCA of which 25 μl are used per reaction. If measuring PP1, PP2A and CN activity, 125 μl per sample are used.
0.75 mM RII phosphopeptide of which 10 μl are used per reaction. If measuring PP1, PP2A and CN activity, 40 μl per sample are used.

### Phosphatase Assay

Remove Pi from RII phosphopeptide by adding PiBind™ resin ( 1/10 of total volume) and incubate on ice for minimum of 10 min with occasional flicking.
Remove Pi from samples by adding 1/10 total volume of PiBind™ resin and incubate on ice for minimum of 10 min with occasional flicking.
Remove PiBind™ resin by centrifugation at 13,000 g for one minute.
Prepare the following in labeled 0.5 ml microcentrifuge tubes (or in a 96-well plate) on ice. The total reaction volume will be 50 μl.

For PP1/PP2A activity:

| | | Resin-treated |
|---|---|---|
| | 1x Assay Buffer 1 | samples |
| Bkgd | 40 μl | 10 μl |
| Total 1 | 30 μl | 10 μl |
| Tautomycin | 30 μl | 10 μl |
| (for PP1) | (with 5 nM Tautomycin) | |
| OA | 30 μl | 10 μl |
| (for PP2A) | (with 16.7 nM OA) | |

For CN activity:

| | | Resin-treated |
|---|---|---|
| | 1x Assay Buffer 2 | samples |
| Total 2 | 30 μl | 10 μl |
| (for CN) | | |

Preheat the samples from step 5 to 30° C. for 10 min in a heating block (or in an incubation chamber for a 96-well plate).
Start the reaction by adding 10 μl resin-treated RII phosphopeptide per reaction (total reaction volume=50 μl).
Incubate at 30° C. for 10 min.
Stop the reaction by adding 25 μl 48% TCA (final reaction concentration=16%) per reaction.
Vortex mix and place on ice for minimum of 5 min.
Centrifuge the tubes (or a plate) at 12,000 g (or maximum) at 4° C. for 5 min.
Aliquot 25 μl MilliQ H₂O into each well of a new 96-well plate at room temperature.
Carefully remove 25 μl of the supernatant of spun-down samples (which contains Pi) and add to each well at room temperature.
Start the colorimetric detection of Pi by adding 100 μl BIOMOL™ Green Reagent into each well.
Incubate for 20 to 30 min at room temperature.
Read OD620 nm on a plate reader.
Calculate the PP1, PP2A and CN activity as follows. First subtract OD₆₂₀ₙₘ Bkgd from all other OD₆₂₀ₙₘ readings. Then calculate the specific activities:

$$PP 1 activity = \frac{100 \% \times ( {OD}_{620 nm} Total 1 - {OD}_{620 nm} Tautomycin )}{{OD}_{620 nm} Total 1}$$

$$PP 2 A activity = \frac{100 \% \times ( {OD}_{620 nm} Total 1 - {OD}_{620 nm} OA )}{{OD}_{620 nm} Total 1}$$

$$CN activity = \frac{100 \% \times ( {OD}_{620 nm} Total 2 - {OD}_{620 nm} Total 1 )}{{OD}_{620 nm} Total 2}$$

The following example demonstrates how PP1, PP2A, and CN activity can be calculated given the following OD₆₂₀ₙₘ measurements:

| | OD₆₂₀ₙₘ | OD₆₂₀₀ₙₘ-Bkgd |
|---|---|---|
| Bkgd | 0.1 | 0 |
| Total 1 | 0.25 | 0.15 |
| 3 nM Tautomycin | 0.2 | 0.1 |
| 10 nM OA | 0.15 | 0.05 |
| Total 2 | 0.35 | 0.25 |

If PP1/PP2A activity is measured separately from CN activity, the following calculation can be done using the (OD₆₂₀ₙₘ-Bkgd) values accordingly to the equations provided above:

PP1 activity=(0.15−0.1)/0.15*100=33.3%

PP2A activity=(0.15−0.05)/0.15*100=66.7%

If PP1, PP2A and CN activity are measured at the same time, Total 2 is used to calculate the PP1 and PP2A activity instead of Total 1. This will give a total activity of 100%, including all three phosphatases. For example:

PP1 activity=(0.15−0.1)/0.25*100=20%

PP2A activity=(0.15−0.05)/0.25*100=40%

CN activity=(0.25−0.15)/0.25*100=40%

The assay of the present invention has been verified and validated with crude subcellular preparation from brain tissue and brain slice homogenate preparation.

### Example 8

### Lentiviral-Mediated Overexpression of PP1 Reduces Ischemic Vulnerability in Organotypic Hippocampal Slices

The function of infected neurons is generally compared to one of uninfected control neurons as well as to one of neurons infected with a control virus to determine the effect of the expressed transgene. That is the reason why the CA1 pyramidal cell layer was infected in vitro with either aCSF (non-infected slices: ni slices), a lentiviral vector expressing PP1 (lenti-PP1 slice), PP1 and green fluorescent protein (GFP) (lenti-EGFP-PP1 slice), or GFP alone (control slice). Cells expressing GFP fluorescence were analyzed by immunostaining with GFP (GFP) and neuronal markers (NeuN) antibody. A merged of both staining was performed ten days after the injections of the lentiviruses, and a highly heterogenous expression in CA1 neurons was observed. To confirm the overexpression of PP1, PP1 mRNA expression in the hippocampus of lenti EGFP-PP1 slice was quantified. It was observed that PP1 mRNA was significantly increased in lenti-EGFP-PP1 slices compared to control slices.

To reproduce conditions of transient brain ischemia in vitro, organotypic hippocampal slices from adult mouse brain were exposed to hypoxia/aglycemia by perfusion with artificial cerebrospinal fluid (aCSF) deprived of oxygen and glucose (OGD) (replaced by nitrogen and sucrose). Under these conditions, in slices, ischemia induced a dramatic drop in slope and amplitude of evoked f-EPSP in CA1 pyramidal neurons. When normoxic aCSF was resumed, f-EPSP partially recovered then stabilized but average f-EPSP slope remained significantly lower than baseline when measured 55 min after ischemia onset (over 15 min) (59.87±0.68 of baseline n=9; p<0.0001, unpaired t-test). Next, it was examined whether the overexpression of PP1 could modify CA1 f-EPSP recovery after transient hypoxia/aglycemia. In control slices, f-EPSP recovery was similar than in ni slices (60.57±0.72 vs. 59.87±0.68 of baseline, one-way ANOVA), the injection of a control virus had no effect on recovery. However, f-EPSP recovery was overall significantly increased after ischemia induction in lenti-PP1 slices (74.21±0.57 n=6; p<0.001, one way ANOVA) or lenti-EGFP-PP1 slices (71.92±0.54, p<0.001, n=11, one way ANOVA) compared to control slices. No significant difference in recovery was observed between lenti-PP1 slice or lenti-EGFP-PP1 suggesting that the effect most likely results from PP1 overexpression, and not from the actual lentiviral infection.

### Example 9

### PP1 Protection Against OGD-Induced Delayed Hippocampal Cell Death

After showing that overexpression of PP1 could modify CA1 f-EPSP recovery after transient hypoxia/aglycemia, it was investigated the effects of OGD severity on neuronal viability under different conditions. First, it was verified that simulating transient ischemia by OGD in the hippocampus in vitro induces delayed neuronal death as occurs in vivo (Schmidt-Kastner and Freund, 1991; Lipton, 1999). Cell death was assayed by adding PI to hippocampal slice cultures. PI rapidly penetrates cells with damaged membrane and fluoresces upon binding to nucleic acids (Macklis and Madison, 1990). OGD for 4 min at 35° C. did not result in immediately detectable PI staining. However, after re-incubation in normal culture medium for 48 h, OGD treatment induced a significant increase in PI staining in the pyramidal layers of CA1. In contrast, OGD-induced PI uptake in Lenti-PP1 slices was reversed and delayed neuronal death was prevented. Thus, these experiments clearly demonstrated that PP1 overexpression prevent OGD-induced delayed neuronal death in CA1 area.

In summary, the findings in the preceding experiments could be confirmed by using the in vitro model of ischemia in which hippocampal slices are transiently deprived of oxygen and glucose (OGD), demonstrating that lentiviral-mediated PP1 overexpression confers a full recovery and tissue protection.

### REFERENCES

Aarts, M. M., and Tymianski, M. (2003). Novel treatment of excitotoxicity: targeted disruption of intracellular signalling from glutamate receptors. Biochem Pharmacol 66, 877-886.
Aggen, J. B., Nairn, A. C. and Chamberlin, R. (2000). Regulation of protein phosphatase-1. Chem. Biol. 7, 13-23.
Alessandrini, A., Namura, S., Moskowitz, M. A., and Bonventre, J. V. (1999). MEK1 protein kinase inhibition protects against damage resulting from focal cerebral ischemia. Proc Natl Acad Sci USA 96, 12866-12869.
Aronowski, J., Grotta, J. C., Strong, R., and Waxham, M. N. (2000). Interplay between the gamma isoform of PKC and calcineurin in regulation of vulnerability to focal cerebral ischemia. J Cereb Blood Flow Metab 20, 343-349.
Arundine, M., and Tymianski, M. (2003). Molecular mechanisms of calcium-dependent neurodegeneration in excitotoxicity. Cell Calcium 34, 325-337.
Arundine, M., and Tymianski, M. (2004). Molecular mechanisms of glutamate-dependent neurodegeneration in ischemia and traumatic brain injury. Cell Mol Life Sci 61, 657-668.
Asai, A., Qiu, J., Narita, Y., Chi, S., Saito, N., Shinoura, N., Hamada, H., Kuchino, Y., and Kirino, T. (1999). High level calcineurin activity predisposes neuronal cells to apoptosis. J Biol Chem 274, 34450-34458.
Ayllon, V., Martinez, A. C., Garcia, A., Cayla, X., and Rebollo, A. (2000). Protein phosphatase 1alpha is a Ras-activated Bad phosphatase that regulates interleukin-2 deprivation-induced apoptosis. Embo J 19, 2237-2246.
Blitzer, R. D., Connor, J. H., Brown, G. P., Wong, T., Shenolikar, S., Iyengar, R., and Landau, E. M. (1998). Gating of CaMKII by cAMP-regulated protein phosphatase activity during LTP. Science 280, 1940-1942.
Bogoyevitch, M. A., Boehm, I., Oakley, A., Ketterman, A. J., and Barr, R. K. (2004). Targeting the JNK MAPK cascade for inhibition: basic science and therapeutic potential. Biochim Biophys Acta 1697, 89-101.
Borsello, T., Clarke, P. G., Hirt, L., Vercelli, A., Repici, M., Schorderet, D. F., Bogousslavsky, J., and Bonny, C. (2003). A peptide inhibitor of c-Jun N-terminal kinase protects against excitotoxicity and cerebral ischemia. Nat Med 9, 1180-1186.
Brichese, L., Cazettes, G., and Valette, A. (2004). JNK is associated with Bcl-2 and PP1 in mitochondria: paclitaxel induces its activation and its association with the phosphorylated form of Bcl-2. Cell Cycle 3, 1312-1319.
Brown, G. P., Blitzer, R. D., Connor, J. H. Wong, T., Shenolikar, S., Iyengar, R., and Landau, E. M. (2000). Long-term potentiation induced by theta frequency stimulation is regulated by a protein phosphatase-1-operated gate. J Neurosci 20, 7880-7887.
Calabresi, P., Centonze, D., Pisani, A., Cupini, L., and Bernardi, G. (2003). Synaptic plasticity in the ischaemic brain. Lancet Neurol 2, 622-629.
Ceulemans, H., and Bollen, M. (2004). Functional diversity of protein phosphatase-1, a cellular economizer and reset button. Physiol Rev 84, 1-39.
Chu, C. T., Levinthal, D. J., Kulich, S. M., Chalovich, E. M., and DeFranco, D. B. (2004). Oxidative neuronal injury. The dark side of ERK1/2. Eur J Biochem 271, 2060-2066.
Cohen, P., Holmes, C. F., and Tsukitani, Y. (1990). Okadaic acid: a new probe for the study of cellular regulation. Trends Biochem Sci 15, 98-102.
Colbran, R. J. (2004). Protein phosphatases and calcium/calmodulin-dependent protein kinase II-dependent synaptic plasticity. J Neurosci 24, 8404-8409.
Farrokhnia, N., Roos, M. W., Terent, A., and Lennmyr, F. (2005). Differential early mitogen-activated protein kinase activation in hyperglycemic ischemic brain injury in the rat. Eur J Clin Invest 35, 457-463.
Fernandez, M. T., Zitko, V., Gascon, S., Torreblanca, A., and Novelli, A. (1993). Neurotoxic effect of okadaic acid, a seafood-related toxin, on cultured cerebellar neurons. Ann N Y Acad Sci 679, 260-269.
Fladmark, K. E., Brustugun, O. T., Hovland, R., Boe, R., Gjertsen, B. T., Zhivotovsky, B., and Doskeland, S. O. (1999). Ultrarapid caspase-3 dependent apoptosis induction by serine/threonine phosphatase inhibitors. Cell Death Differ 6, 1099-1108.
Fukunaga, K., Muller, D., Ohmitsu, M., Bako, E., DePaoli-Roach, A. A., and Miyamoto, E. (2000). Decreased protein phosphatase 2A activity in hippocampal long-term potentiation. J Neurochem 74, 807-817.
Gao, T. M., Pulsinelli, W. A., and Xu, Z. C. (1998). Prolonged enhancement and depression of synaptic transmission in CA1 pyramidal neurons induced by transient forebrain ischemia in vivo. Neuroscience 87, 371-383.
Genoux, D., Haditsch, U., Knobloch, M., Michalon, A., Storm, D., and Mansuy, I. M. (2002). Protein phosphatase 1 is a molecular constraint on learning and memory. Nature 418, 970-975.
Guan, Q. H., Pei, D. S., Xu, T. L., and Zhang, G. Y. (2006). Brain ischemia/reperfusion-induced expression of DP5 and its interaction with Bcl-2, thus freeing Bax from Bcl-2/Bax dimmers are mediated by c-Jun N-terminal kinase (JNK) pathway. Neurosci Lett 393, 226-230.
Gupta, V., Ogawa, A. K., Du, X., Houk, K. N., and Armstrong, R. W. (1997). A model for binding of structurally diverse natural product inhibitors of protein phosphatases PP1 and PP2A. J Med Chem 40, 3199-3206.
Hermann, D. M., Kilic, E., Kugler, S., Isenmann, S., and Bahr, M. (2001). Adenovirus-mediated GDNF and CNTF pretreatment protects against striatal injury following transient middle cerebral artery occlusion in mice. Neurobiol Dis 8, 655-666.
Hetman, M., and Gozdz, A. (2004). Role of extracellular signal regulated kinases 1 and 2 in neuronal survival. Eur J Biochem 271, 2050-2055.
Hori, N., and Carpenter, D. O. (1994). Functional and morphological changes induced by transient in vivo ischemia. Exp Neurol 129, 279-289.
Horiguchi, T., Shima, H., Suga, S., Ogino, M., Shimizu, K., Toya, S., Nagao, M., and Kawase, T. (2002). Transient forebrain ischemia induces expression of serine/threonine protein phosphatase 1 mRNA in the vulnerable regions of gerbil brain. Neurosci Lett 325, 115-118.
Hoyte, L., Barber, P. A., Buchan, A. M., and Hill, M. D. (2004). The rise and fall of NMDA antagonists for ischemic stroke. Curr Mol Med 4, 131-136.
Irving, E. A., and Bamford, M. (2002). Role of mitogen- and stress-activated kinases in ischemic injury. J Cereb Blood Flow Metab 22, 631-647.
Jouvenceau, A., Hedou, G., Potier, B., Kollen, M., Dutar, P., and Mansuy, I. M. (2006). Partial inhibition of PP1 alters bidirectional synaptic plasticity in the hippocampus. Eur J Neurosci 24, 564-572.
Kaminska, B., Gaweda-Walerych, K., and Zawadzka, M. (2004). Molecular mechanisms of neuroprotective action of immunosuppressants—facts and hypotheses. J Cell Mol Med 8, 45-58.
Katsura, K. I., Kurihara, J., Kato, H., and Katayama, Y. (2001). Ischemic pre-conditioning affects the subcellular distribution of protein kinase C and calcium/calmodulin-dependent protein kinase II in the gerbil hippocampal CA1 neurons. Neurol Res 23, 751-754.
Kilic, E., Kilic, U., Soliz, J., Bassetti, C. L., Gassmann, M., and Hermann, D. M. (2005). Brain-derived erythropoietin protects from focal cerebral ischemia by dual activation of ERK-1/-2 and Akt pathways. Faseb J 19, 2026-2028.
Klettner, A., Baumgrass, R., Zhang, Y., Fischer, G., Burger, E., Herdegen, T., and Mielke, K. (2001). The neuroprotective actions of FK506 binding protein ligands: neuronal survival is triggered by de novo RNA synthesis, but is independent of inhibition of JNK and calcineurin. Brain Res Mol Brain Res 97, 21-31.
Klumpp, S., and Krieglstein, J. (2002). Serine/threonine protein phosphatases in apoptosis. Curr Opin Pharmacol 2, 458-462.
Legos, J. J., Tuma, R. F., and Barone, F. C. (2002). Pharmacological interventions for stroke: failures and future. Expert Opin Investig Drugs 11, 603-614.
Lipton, P. (1999). Ischemic cell death in brain neurons. Physiol Rev 79, 1431-1568.
Lisman, J. E., and Zhabotinsky, A. M. (2001). A model of synaptic memory: a CaMKII/PP1 switch that potentiates transmission by organizing an AMPA receptor anchoring assembly. Neuron 31, 191-201.
Lobner, D., and Lipton, P. (1993). Intracellular calcium levels and calcium fluxes in the CA1 region of the rat hippocampal slice during in vitro ischemia: relationship to electrophysiological cell damage. J Neurosci 13, 4861-4871.
Lu, Y. M., Mansuy, I. M., Kandel, E. R., and Roder, J. (2000). Calcineurin-mediated LTD of GABAergic inhibition underlies the increased excitability of CA1 neurons associated with LTP. Neuron 26, 197-205.
Lust, W. D., Taylor, C., Pundik, S., Selman, W. R., and Ratcheson, R. A. (2002). Ischemic cell death: dynamics of delayed secondary energy failure during reperfusion following focal ischemia. Metab Brain Dis 17, 113-121.
MacKintosh, C., and Klumpp, S. (1990). Tautomycin from the bacterium Streptomyces verticillatus. Another potent and specific inhibitor of protein phosphatases 1 and 2A. FEBS Lett 277, 137-140.
Macklis J D, Madison R D (1990) Progressive incorporation of propidium iodide in cultured mouse neurons correlates with declining electrophysiological status: a fluorescence scale of membrane integrity. J Neurosci Methods 31:43-46.
Manfroid, I., Martial, J. A., and Muller, M. (2001). Inhibition of protein phosphatase PP1 in GH3B6, but not in GH3 cells, activates the MEK/ERK/c-fos pathway and the human prolactin promoter, involving the coactivator CPB/p300. Mol Endocrinol 15, 625-637.
Mansuy, I. M., and Shenolikar, S. (2006). Protein serine/threonine phosphatases in neuronal plasticity and disorders of learning and memory. Trends Neurosci 29, 679-686.
Morioka, M., Hamada, J., Ushio, Y., and Miyamoto, E. (1999). Potential role of calcineurin for brain ischemia and traumatic injury. Prog Neurobiol 58, 1-30.
Morishita, W., Connor, J. H., Xia, H., Quinlan, E. M., Shenolikar, S., and Malenka, R. C. (2001). Regulation of synaptic strength by protein phosphatase 1. Neuron 32, 1133-1148.
Nuydens, R., de Jong, M., Van Den Kieboom, G., Heers, C., Dispersyn, G., Cornelissen, F., Nuyens, R., Borgers, M., and Geerts, H. (1998). Okadaic acid-induced apoptosis in neuronal cells: evidence for an abortive mitotic attempt. J Neurochem 70, 1124-1133.
Olney, J. W., and Sharpe, L. G. (1969). Brain lesions in an infant rhesus monkey treated with monsodium glutamate. Science 166, 386-388.
Onodera, H., Yamasaki, Y., Kogure, K., and Miyamoto, E. (1995). Calcium/calmodulin-dependent protein kinase II and protein phosphatase 2B (calcineurin) immunoreactivity in the rat hippocampus long after ischemia. Brain Res 684, 95-98.
Papas, S., Crepel, V., and Ben-Ari, Y. (1993). The NMDA receptor contributes to anoxic aglycemic induced irreversible inhibition of synaptic transmission. Brain Res 607, 54-60.
Quevedo, C., Salinas, M., and Alcazar, A. (2003). Initiation factor 2B activity is regulated by protein phosphatase 1, which is activated by the mitogen-activated protein kinase-dependent pathway in insulin-like growth factor 1-stimulated neuronal cells. J Biol Chem 278, 16579-16586.
Raley-Susman, K. M., and Lipton, P. (1990). In vitro ischemia and protein synthesis in the rat hippocampal slice: the role of calcium and NMDA receptor activation. Brain Res 515, 27-38.
Runden, E., Seglen, P. O., Haug, F. M., Ottersen, O. P., Wieloch, T., Shamloo, M., and Laake, J. H. (1998). Regional selective neuronal degeneration after protein phosphatase inhibition in hippocampal slice cultures: evidence for a MAP kinase-dependent mechanism. J Neurosci 18, 7296-7305.
Sattler, R., and Tymianski, M. (2000). Molecular mechanisms of calcium-dependent excitotoxicity. J Mol Med 78, 3-13.
Schmidt-Kastner R, Freund T F (1991) Selective vulnerability of the hippocampus in brain ischemia. Neuroscience 40:599-636.
Shackelford, D. A., and Yeh, R. Y. (2006). Modulation of ERK and JNK activity by transient forebrain ischemia in rats. J Neurosci Res 83, 476-488.
Sharkey, J., and Butcher, S. P. (1994). Immunophilins mediate the neuroprotective effects of FK506 in focal cerebral ischaemia. Nature 371, 336-339.
Sharkey, J., Crawford, J. H., Butcher, S. P., and Marston, H. M. (1996). Tacrolimus (FK506) ameliorates skilled motor deficits produced by middle cerebral artery occlusion in rats. Stroke 27, 2282-2286.
Spudich, A., Kilic, E., Xing, H., Kilic, U., Rentsch, K. M., Wunderli-Allenspach, H., Bassetti, C. L., and Hermann, D. M. (2006). Inhibition of multidrug resistance transporter-1 facilitates neuroprotective therapies after focal cerebral ischemia. Nat Neurosci 9, 487-488.
Stevens, T. R., Krueger, S. R., Fitzsimonds, R. M., and Picciotto, M. R. (2003). Neuroprotection by nicotine in mouse primary cortical cultures involves activation of calcineurin and L-type calcium channel inactivation. J Neurosci 23, 10093-10099.
Sugawara, T., Fujimura, M., Noshita, N., Kim, G. W., Saito, A., Hayashi, T., Narasimhan, P., Maier, C. M., and Chan, P. H. (2004). Neuronal death/survival signaling pathways in cerebral ischemia. NeuroRx I, 17-25.
Thiels, E., Norman, E. D., Barrionuevo, G., and Klann, E. (1998). Transient and persistent increases in protein phosphatase activity during long-term depression in the adult hippocampus in vivo. Neuroscience 86, 1023-1029.
Wang, H. G., Pathan, N., Ethell, I. M., Krajewski, S., Yamaguchi, Y., Shibasaki, F., McKeon, F., Bobo, T., Franke, T. F., and Reed, J. C. (1999). Ca2+-induced apoptosis through calcineurin dephosphorylation of BAD. Science 284, 339-343.
Wang, Y., Kilic, E., Kilic, U., Weber, B., Bassetti, C. L., Marti, H. H., and Hermann, D. M. (2005). VEGF overexpression induces post-ischaemic neuroprotection, but facilitates haemodynamic steal phenomena. Brain 128, 52-63.
Westphal, R. S., Tavalin, S. J., Lin, J. W., Alto, N. M., Fraser, I. D., Langeberg, L. K., Sheng, M., and Scott, J. D. (1999). Regulation of NMDA receptors by an associated phosphatase-kinase signaling complex. Science 285, 93-96.
Winder, D. G., and Sweatt, J. D. (2001). Roles of serine/threonine phosphatases in hippocampal synaptic plasticity. Nat Rev Neurosci 2, 461-474.
Yi, K. D., Chung, J., Pang, P., and Simpkins, J. W. (2005). Role of protein phosphatases in estrogen-mediated neuroprotection. J Neurosci 25, 7191-7198.
Youssef, F. F., Addae, J. I., McRae, A., and Stone, T. W. (2001). Long-term potentiation protects rat hippocampal slices from the effects of acute hypoxia. Brain Res 907, 144-150.
Youssef, F. F., Addae, J. I., and Stone, T. W. (2003). LTP-induced depression of response to hypoxia in hippocampus: effects of adenosine receptor activation. Neuroreport 14, 1809-1814.
Youssef, F. F., Addae, J. I., and Stone, T. W. (2006). NMDA-induced preconditioning attenuates synaptic plasticity in the rat hippocampus. Brain Res 1073-1074, 183-189.

1. An agent capable of maintaining, restoring or promoting phosphatase activity of protein phosphatase 1 (PP1) for the treatment, amelioration or prevention of an acute cerebral neurological condition.

2. The agent of claim 1, wherein said neurological condition is due to an excitotoxic injury.

3. The agent of claim 1, wherein said neurological condition is selected from the group consisting of stroke, head or spinal cord trauma, seizure, or neurodegenerative disorders.

4. The agent of claim 1, wherein said neurological condition is acute ischemic insult.

5. The agent of claim 1, wherein said agent is capable of inducing long-term depression (LTD).

6. The agent of claim 5, which is low frequency stimulation (LFS).

7. The agent of, wherein said agent is a PP1-activator/agonist.

8. The agent of claim 7, wherein said agent is selected from the group consisting of ceramide, inhibitor-2, cdc2-cyclin B, and glycogen synthase kinase 3 (GSK3).

9. A method of diagnosis of an acute neurological condition said method comprising:
(a) assaying a sample from a subject for PP1 gene product or activity; and
(b) determining the level of PP1 gene product or activity,
wherein an altered level compared to a control indicates the presence of the condition.

10. (canceled)

11. A kit for the non-radioactive detection of the activity of PP1, PP2A, and calcineurin (PP2B), comprising:
(a) an inhibitor that preferentially inhibits either PP1 alone or PP2A alone;
(b) a compound capable of interfering with protein kinase A activity and blocking phosphorylation-dependent endogenous phosphatase inhibitors;
(c) a compound capable of blocking calcineurin activity; and optionally
(d) a phosphatase substrate.

12. The kit of claim 11, wherein said inhibitor of (a) is tautomycin and/or okadaic acid, said compound of (b) is caffeine, said compound of (c) is EDTA, and/or said substrate is RII phosphopeptide.

13. The kit of claim 11, further comprising calmodulin.

14. The kit of claim 11, further comprising TCA.

15. The kit of claim 11, further comprising a PiBind desalting resin.

16. The kit of claim 11, further comprising a means for colorimetric detection of inorganic phosphates.

17. The kit of claim 12, further comprising calmodulin.

18. The kit of claim 12, further comprising TCA.

19. The kit of claim 13, further comprising TCA.

20. The kit of claim 12, further comprising a PiBind desalting resin.

21. The kit of claim 13, further comprising a PiBind desalting resin.
